(19) United States
(12) Patent Application Publication
(10) Pub. No.: US 2023/0203099 A1
(43) Pub. Date: Jun. 29, 2023

(54) CYCLOPHILIN INHIBITORS AND USES THEREOF

(71) Applicant: Farsight Medical Technology (Shanghai) Co., Ltd., Shanghai (CN)

(72) Inventors: Ching-Pong Mak, Shanghai (CN); Hans Fliri, Shanghai (CN); Fashu Ma, Shanghai (CN); Michael Peel, Shanghai (CN)

(21) Appl. No.: 17/906,978

(22) PCT Filed: Mar. 25, 2021
(86) PCT No.: PCT/CN2021/083015

§ 371 Date: Sep. 22, 2022

(30) Foreign Application Priority Data
Mar. 26, 2020 (WO) PCT/CN2020/081296

(51) Int. Cl. C07K 7/64; A61K 38/13; A61K 9/00

(52) U.S. Cl. CPC C07K 7/645; A61K 38/13; A61K 9/0019

(57) ABSTRACT

Provided are compounds as defined by Formula 1 and uses thereof for the prevention or treatment of disease or conditions such as organ injury or organ failure.

### BACKGROUND OF THE INVENTION

The present invention relates to cyclosporin analogues, and their use for the treatment or prevention of disease or disorders, in particular disease or conditions associated with cellular injury or cell death, which can be caused by a number of different reasons, such as ischemia or ischemia reperfusion injury, or toxins, infection or mechanical trauma. In particular, the invention relates to compounds which may be provided as potent cyclophilin D inhibitors.

Acute inflammation is well recognized to involve the complex interaction of various cellular (neutrophils, macrophages) and extracellular (complement, histamine) factors that act in response to PAMP (pathogen-activated molecular patterns) and DAMP (damage-activated molecular patterns) signals to resolve the originating insult. Cyclophilin A has been demonstrated to function as a chemokine to facilitate leukocyte migration in support of an inflammatory response and blockade of cyclophilin A was shown to be beneficial in animal models of acute inflammation. More recently a severe form of inflammation that is accompanied by cell death and tissue necrosis has been described. A significant body of evidence now supports the opening of a pore at the mitochondrial membrane, termed the Mitochondrial Permeability Transition Pore (MPTP), as being critical to the onset and maintenance of this necrotic inflammation. A key regulator of this MPTP opening is Cyclophilin D (CypD), and inhibitors of CypD have shown good activity in preventing tissue damage associated with necrotic inflammation. Opening of the MPTP, and subsequent initiation of necrotic cell death, is triggered by elevated intracellular calcium levels that result from a variety of factors including excessive physiological signals (e.g. noise trauma, excitotoxicity), oxidative stress, hypoxia, bile salt toxins, etc. Notably, genetic ablation, or pharmacological inhibition, of CypD was found to be protective toward tissue degradation due to ischemia-reperfusion injury of cardiac tissue suggesting that CypD inhibition is a viable drug target for ischemia-reperfusion injury more generally. In mouse models cyclophilin D deletion was shown to have a significant protective effect against damage caused to the kidney by a severe ischemia-reperfusion insult (Am J Physiol Renal Physiol 297: F749-F759, 2009). This protective effect was evident in improved renal function, as measured by serum creatinine levels, and in tissue damage (measured by histology) in the cyclophilin D knockout animals compared to wild type controls. Similarly, in a mouse model of kidney damage caused by administration of a nephrotoxic drug (Am J Physiol Renal Physiol. 2019 Sep. 1; 317(3):F683-F694), animals in which cyclophilin D was knocked out were more resistant to oxidative stress and hypomethylation and had lower indications of renal toxicity than wild type animals.

In studies carried out using cyclophilin D knockout mice as well as pharmacological strategies with cyclophilin inhibitors it has been unambiguously demonstrated that opening of the mitochondrial permeability transition pore (MPTP), a non-specific channel in the inner mitochondrial membrane, is a fundamental event in cell death that results from a variety of insults. Further, inhibition of cyclophilin D can prevent opening of the mPTP which is protective toward mitochondrial function and preserves cell viability.

Cyclosporin A is a compound well known for its immunosuppressive properties, but other biological properties have also been described. Cyclosporin A has the following chemical structure:

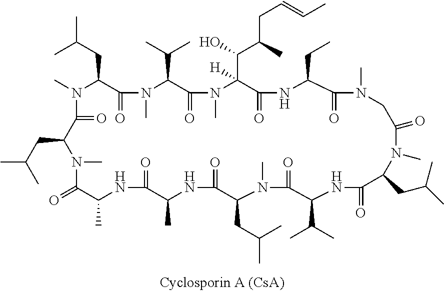 [SMILES: C/C=C/C[C@@H](C)[C@@H](O)[C@H]1C(=O)N[C@@H](CC)C(=O)N(C)CC(=O)N(C)[C@@H](CC(C)C)C(=O)N[C@@H](C(C)C)C(=O)N(C)[C@@H](CC(C)C)C(=O)N[C@@H](C)C(=O)N[C@H](C)C(=O)N(C)[C@@H](CC(C)C)C(=O)N(C)[C@@H](CC(C)C)C(=O)N(C)[C@@H](C(C)C)C(=O)N1C]

Biologically active derivatives of Cyclosporin A have also been made. For example, U.S. Pat. No. 6,583,265, EP0484281, EP0194972 describes cyclosporin derivatives having various properties including immunosuppressive, antiparasitic and antiviral properties. Further examples include, U.S. Pat. No. 6,809,077 which describes cyclosporin derivatives with modifications made at position 1, and methods of treatment comprising said compounds such as for prevention of organ transplantation rejection and treatment of autoimmune diseases in subjects.

U.S. Pat. No. 6,583,265 describes cyclosporin derivatives with modifications made at position 3 of the cyclosporin macrocycle. In particular, U.S. Pat. No. 6,583,265 discloses Compound 1:

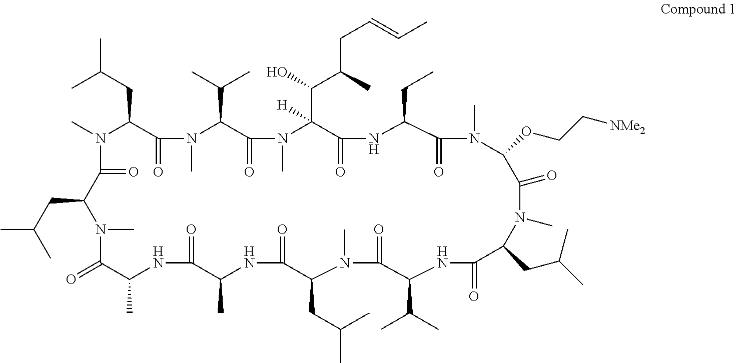 [SMILES: C/C=C/C[C@@H](C)[C@@H](O)[C@H]1C(=O)N[C@@H](CC)C(=O)N(C)[C@H](OCCN(C)C)C(=O)N(C)[C@@H](CC(C)C)C(=O)N[C@@H](C(C)C)C(=O)N(C)[C@@H](CC(C)C)C(=O)N[C@@H](C)C(=O)N[C@H](C)C(=O)N(C)[C@@H](CC(C)C)C(=O)N(C)[C@@H](CC(C)C)C(=O)N(C)[C@@H](C(C)C)C(=O)N1C]

WO2019/016572 A1 also describes Compound 1 for use in the treatment or prevention of acute or chronic inflammatory disorders.

It is an object of the present invention to provide further cyclosporin analogues, in particular analogues which may be useful for inhibition of cyclophilins, e.g. cyclophilin A, B, D, and diseases and conditions associated therewith. Further objects of the invention will be clear on the basis of the following description of the invention, examples and claims.

### SUMMARY OF THE INVENTION

In a first aspect, the invention relates to a compound of Formula 1,

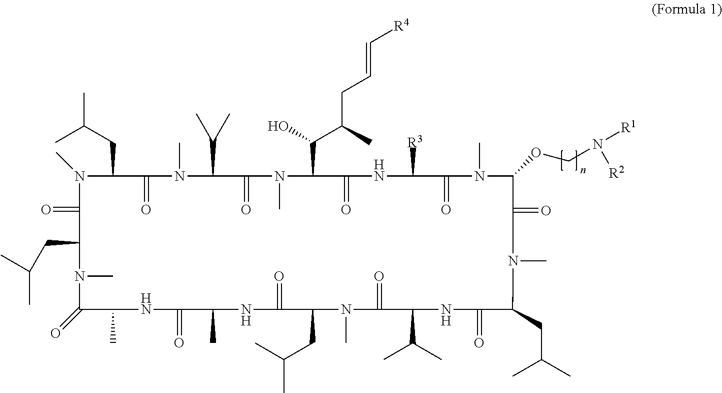

or a pharmaceutically acceptable salt thereof, wherein:

n is selected from an integer between 2 and 5; R¹ and R² are independently selected from H, C₁ to C₆ alkyl or wherein R₁ and R₂ may be joined together to form a C₃ to C₆ cycloalkyl or heterocycloalkyl ring; R³ is ethyl, 1-hydroxyethyl, isopropyl, or n-propyl; and wherein R⁴ is aryl, substituted aryl, heteroaryl and substituted heteroaryl, and wherein the substitution is optionally one or more substituents independently selected from C₁ to C₆ alkyl, halogen, haloalkyl, hydroxyl, C₁ to C₆ alkoxyl, amino, monoalkylamino, dialkylamino, thioalkyl, nitro, cyano, carboxyl, alkoxycarbonyl, aryl and heteroaryl.

In a related aspect, the invention provides for a compound of Formula 2

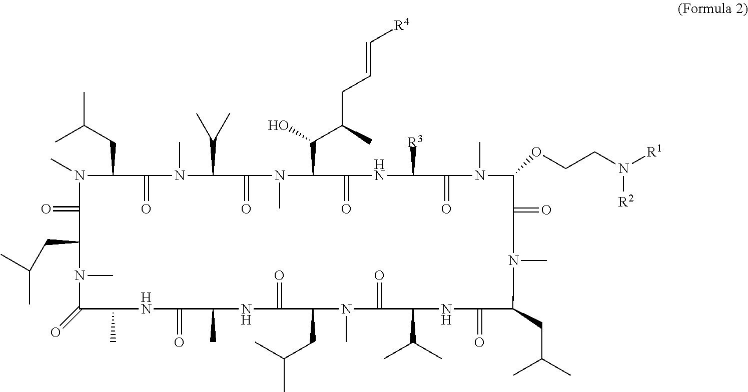

as depicted wherein R¹ and R², R³ and R⁴ may be defined as for Formula 1, or specific embodiments thereof.

In further aspect, the invention provides for use of said compounds as cyclophilin inhibitors. In yet a further aspect, the invention provides for use of said compounds in a method of treating or preventing diseases or condition associated with cell injury or cell death, such as organ injury or organ failure.

### DETAILED DESCRIPTION OF THE INVENTION

The present invention relates, in a first aspect, to a compound of Formula 1, or a pharmaceutically acceptable salt thereof,

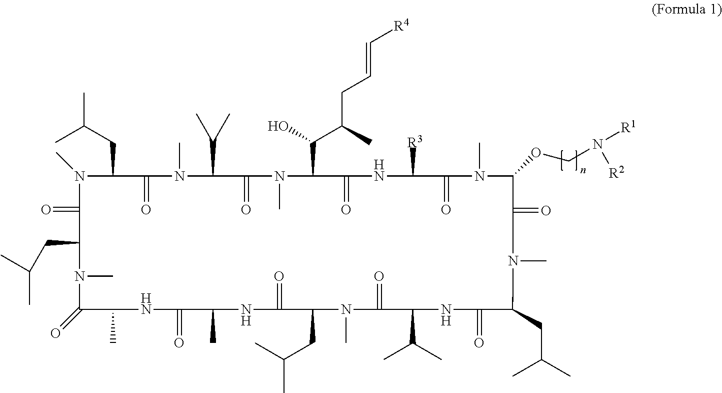

wherein:

n is selected from an integer between 2 and 5;

R¹ and R² are independently selected from H, C₁ to C₆ alkyl or wherein R¹ and R² may be joined together to form a C₃ to C₆ cycloalkyl or heterocycloalkyl ring;

R³ is ethyl, 1-hydroxyethyl, isopropyl, or n-propyl; and wherein R⁴ is aryl, substituted aryl, heteroaryl and substituted heteroaryl, and wherein the substitution is optionally one or more substituents independently selected from C₁ to C₆ alkyl, halogen, haloalkyl, hydroxyl, C₁ to C₆ alkoxyl, amino, monoalkylamino, dialkylamino, thioalkyl, nitro, cyano, carboxyl, alkoxycarbonyl, aryl and heteroaryl.

The cyclosporin compound according to the present disclosure is a cyclosporin A compound comprising an amino alkoxy substituent at the sarcosine residue at position 3 and derivatives of MeBmt at position 1 of the macrocycle, for example as depicted and defined for Formula 1 above. The position numbering as used herein in reference to cyclosporin macrocycle refers to commonly used nomenclature and number assignment of the 11 amino acid residues featured in the cyclosporin core. With cyclosporin A as basis, the amino acids residues may be numbered as follows: methyl-butenyl-threonine, which may be abbreviated as MeBmt (1), aminobutyric acid (2)), sarcosine, which may be abbreviated as Sar (3), N-methyl leucine (4), valine (5), N-methyl leucine (6), alanine (7), D-alanine (8), N-methyl leucine (9), N-methyl leucine (10), N-methyl valine (11).

In one embodiment, the amino alkoxy sarcosine substituent is as depicted in Formula 1. In an optional embodiment, the hydrogen atoms of the alkoxy (i.e. —(CH₂)ₙ—) moiety of this substituent may also be independently replaced with a substituent, such as an alkyl substituent (e.g. methyl), or another substituent such as described herein. For example, the amino alkoxy sarcosine substituent may be a 1-amino-2 methyl-2-propanoyl substituent.

The term ‘H’ as used herein refers to hydrogen.

The term ‘C₁ to C₆ alkyl’ as used herein is defined as saturated or unsaturated alkyl hydrocarbon moiety comprising 1 to 6 carbon atoms in any isomeric configuration. Included are straight-chain, linear alkyl, such as methyl, ethyl, n-propyl, n-butyl, 1-pentyl, n-hexyl. Also included are branched alkyl (i.e. branched C₃ to C₆ alkyl) such as isopropyl, sec-butyl, isobutyl, tert-butyl, 2-pentyl, 3-pentyl, isopentyl, tert-pentyl, neopentyl, and isomers of hexyl. Further included within the definition of ‘C₁ to C₆ alkyl’ are cyclic isomers such as cyclopropyl, cyclobutyl, cyclopentyl, and cyclohexyl. Examples of unsaturated C₁ to C₆ alkyl include but are not limited to vinyl, allyl, butenyl, pentenyl, and hexenyl, and other alkenyl or alkylene moieties, for example comprising one or more double bonds e.g. pentadienyl. The term ‘C₃ to C₆’ alkyl is to be understood analogously but denoting a moiety comprising a range of 3 to 6 carbon atoms. In preferred embodiments, the alkyl substituent is an unsubstituted hydrocarbon moiety such as defined above. In an optional embodiment, the C₁ to C₆ alkyl may be substituted with one or more substituents such as defined below, where by one or more hydrogen atoms are replaced with a bond to said substituent.

In one embodiment, at least one of R¹ or R² of Formula 1 is C₁ to C₆ alkyl. In another embodiment, both of R¹ and R² are —CH₃ (methyl). In one particular embodiment, n is 2, and both of R¹ and R² are —CH₃ (methyl).

In some embodiments, two adjacent R¹ and R² substituents may be joined together so as to form a ring together, for example a C₃ to C₆ cycloalkyl ring. “Cycloalkyl” as used herein is a saturated, or unsaturated non-aromatic hydrocarbon ring. Examples of the moieties formed by adjacent R¹ and R² substituents joining together to form a ring, in the context of the present compounds of Formula 1 or 2, may include azetidine, pyrrolidine, or piperidine. In an optional embodiment, the cycloalkyl moiety may be substituted with one or more substituents such as defined below, where by one or more hydrogen atoms are replaced with a bond to said substituent.

The term ‘hetero’ when used to describe a compound or substituent means that one or more carbon atoms are replaced by a oxygen, nitrogen or sulfur atom. In further embodiments of the current disclosure, the substituents R¹ and R² may be joined together to form a heterocycloalkyl ring, for example a C₃ to C₆ heterocycloalkyl ring. Unless otherwise indicated, ‘heterocycloalkyl’ refers to a saturated, or unsaturated non-aromatic ring forming at least part of a cyclic structure and where at least one or more carbon atoms are replaced by oxygen, nitrogen or sulfur atom (and in the case of a C₃ to C₆ heterocycloalkyl ring, comprising between 3 to 6 carbon atoms). For example, the substituents R¹ and R² may be joined together to form a may be a 4-, 5- or 6-member saturated, non-aromatic ring comprising at least one heteroatom in addition to the nitrogen to which R¹ and R² are joined as featured in Formula 1. The heterocycloalkyl ring may comprise at least one heteroatom selected from O, N, or S.

In one particular embodiment, R¹ and R² are joined together so as to form a morpholine residue. In yet further embodiment, the integer n is 2, and R¹ and R² are joined together so as to form a morpholine residue.

In one embodiment of the invention, R⁴ of the compound as defined by Formula 1 is aryl or substituted aryl.

The term ‘aryl’ as used herein refers to carbocyclic ring system having one (monocyclic) or more (e.g. bicyclic) aromatic rings; examples may include, but are not limited to: phenyl, naphthalenyl, anthracenyl, or the like. In one embodiment, R⁴ is naphthalene, or a substituted naphthalene. The aryl ring radical may be joined to the compound or molecule at any one the ring atoms.

The term ‘substituted’ aryl refers to an aryl moiety or radical, wherein one or more hydrogens are replaced, independently, with at least one or more (e.g. two, three, or more) substituents including, but not limited to C₁ to C₆ alkyl, halogen, haloalkyl, hydroxyl (—OH), C₁ to C₆ alkoxyl, amino (—NH₂), monoalkylamino, dialkylamino, thioalkyl, nitro, cyano, carboxyl, alkoxycarbonyl, aryl and heteroaryl. The substituent(s) may be featured on any one of the ring atoms of the aryl moiety not joined to the compound or molecule.

In another embodiment, R⁴ is aryl (e.g. phenyl, or napthalenyl) substituted with one or more substituents selected from C₁ to C₆ alkyl, hydroxyl, C₁ to C₆ alkoxyl, amino (—NH₂), monoalkylamino, dialkylamino, aryl, and heteroaryl.

The term ‘halogen’ interchangeable with ‘halo’ refers to chloro, bromo, iodo or fluoro atoms. ‘Haloalkyl’ refers to an alkyl substituent wherein one or more hydrogen atoms are replaced by one or more halogen atoms. In one embodiment, the substituent may be a C₁-C₆ haloalkyl, for example, trifluoroalkyl such as trifluoromethyl (—CF₃).

The term ‘hydroxyl’ refers to a —OH radical. In some embodiments, the hydrogen may be substituted, for example with a hydroxy protecting group within the art. The term ‘alkoxyl’ or the like means an alkylated hydroxyl substituent, i.e. in which the hydrogen is replaced by an alkyl group. ‘C₁ to C₆ alkoxyl’ refers to the replacement of hydrogen with a C₁ to C₆ alkyl such as defined above. Examples include methoxy, isopropoxy, phenoxy, or t-butoxy.

The term ‘amino’ refers to an —NH₂ radical. In some embodiments, the hydrogen(s) may be substituted, for example with a protecting group, or one or more further substituent such as alkyl. The term ‘monoalkylamino’, refers to an amino radical in which one of the hydrogens is replaced with alkyl, e.g. C₁ to C₆ alkyl such as defined above (i.e. —NHR, wherein R is alkyl). ‘Dialkylamino’ refers to an amino radical whereby both hydrogens are replaced independently with alkyl (i.e. —NRR′, where R and R′ are alkyl, which may be the same (e.g. dimethylamino), or different).

‘Thioalkyl’ refers to the radical —SR″, wherein R″ is alkyl, C₁ to C₆ alkyl such as defined above. The term ‘carboxyl’ as used herein refers to the radical —C(O)—R^{a}, wherein R^{a} may be selected from hydrogen, alkyl, aryl, hetaryl, hydroxy, alkoxy (e.g. —OCH₃), amino, alkylamino, dialkyl amino, thioalkyl and the like. The term ‘alkoxycarbonyl’ as used herein refers to the radical —OC(O)—R^{a}, wherein R^{a} is selected from alkyl (e.g. C₁ to C₆ alkyl, e.g. methyl), aryl, hetaryl, alkoxy, amino, alkylamino, dialkyl amino, thioalkyl, etc.

In other embodiments, the substitution on a substituted aryl ring may be on adjacent carbon atoms, wherein the substituent moieties are joined to form a ring, such as a cycloalkyl or heterocycloalkyl as defined herein.

In a particular embodiment, R⁴ in the compound of Formula 1 is phenyl, or substituted phenyl wherein the substitution is optionally one or more substituents independently selected from C₁ to C₆ alkyl (e.g. methyl, or t-butyl), halogen (e.g. chloro, fluoro, bromo, or iodo), haloalkyl (e.g. trifluoromethyl), hydroxyl, C₁ to C₆ alkoxyl (e.g. methoxy, phenoxy, or t-butoxy), amino, monoalkylamino, dialkylamino (e.g. dimethylamino), thioalkyl, nitro, cyano, carboxyl (e.g. —COOCH₃), alkoxycarbonyl (e.g. acetoxy), aryl (e.g. phenyl) and heteroaryl.

In a further embodiment, R⁴ is mono-substituted phenyl, for example, a para-substituted phenyl (e.g. p-tolyl, p-methoxyphenyl, p-trifluoromethylphenyl), or alternatively ortho/meta isomers thereof. In alternative embodiments R⁴ is a di-, or tri-substituted phenyl. In yet a further embodiment, R⁴ is phenyl, and substituted with one or more substituents selected from C₁ to C₆ alkyl, hydroxyl, C₁ to C₆ alkoxyl, amino (—NH₂), monoalkylamino, dialkylamino, aryl, and heteroaryl.

In a further embodiment, R⁴ of the compound as defined by Formula 1 is heteroaryl or substituted heteroaryl.

The term ‘heteroaryl’ refers to a cyclic aromatic ring system having one or more (e.g. bicyclic, for example) aromatic rings in which one of the ring atoms is replaced by at least one atom selected from S, O and N, with the remaining atoms being carbon. The cyclic aromatic ring system may for example comprise of five to ten ring atoms and may comprise of one, two or more rings. ‘Substituted heteroaryl’ refers to a heteroaryl moiety wherein one or more hydrogen atoms are replaced, independently, with at least one or more (e.g. two, three, or more) substituents such as defined herein. The heteroaryl radical may be joined to the compound at any of the ring atoms to the compound.

In one embodiment, R⁴ as defined for the compound of Formula 1 is heteroaryl and selected from the group consisting of pyridine, pyrrole, pyrazine, pyrimidine, thiophene, thiazole, oxazole, isoxazole, furan, quinoline, pyrazole, and imidazole, optionally substituted with one or more substituents independently selected from C₁ to C₆ alkyl, halogen, haloalkyl, hydroxyl, C₁ to C₆ alkoxyl, amino, monoalkylamino, dialkylamino, thioalkyl, nitro, cyano, carboxyl, alkoxycarbonyl, aryl and heteroaryl.

In another embodiment of the compound of Formula 1, n is 2 (i.e. providing an −2-disubstituted aminoethoxy radical).

In particular, the invention may relate to a compound of Formula 1, or a pharmaceutically acceptable salt thereof, wherein n is 2, i.e. a compound of Formula 2:

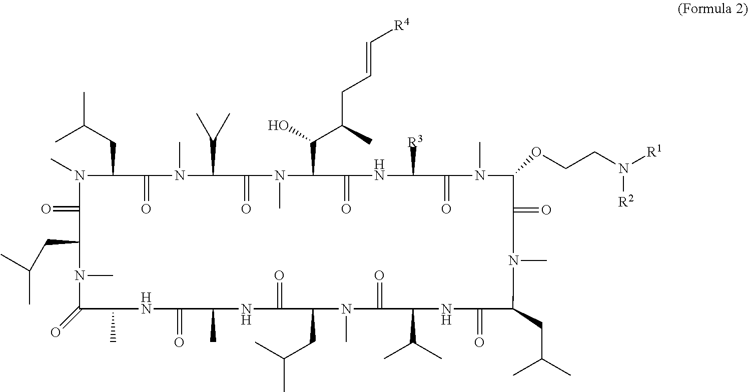

wherein the substituents R¹, R², R³, and R⁴ are defined in accordance with any one or combination of the embodiments or preferences as described above.

In one particular embodiment of the compound of Formula 1, and of Formula 2, R³ is ethyl. In yet further embodiment in respect of a compound of Formula 1, or Formula 2, R³ is ethyl, and R¹ and R² are selected from alkyl, preferably C₁-C₆ alkyl, wherein R¹ and R² may be the same (e.g. both are methyl), or alternatively, wherein R¹ and R² independently selected (i.e. different) C₁-C₆ alkyl. In particular, the invention may relate to a compound of Formula 2, wherein R³ is ethyl, R¹ and R² are both methyl (—CH₃), and R⁴ is as defined in any one of the embodiments described above.

In other embodiments according to the invention, the radical R³ of the compound defined by Formula 1, or Formula 2, is selected from isopropyl, n-propyl, and 1-hydroxyethyl.

In more specific embodiments, the disclosure may relate to a compound, or a pharmaceutically acceptable salt thereof of Formula 2 selected from the following:

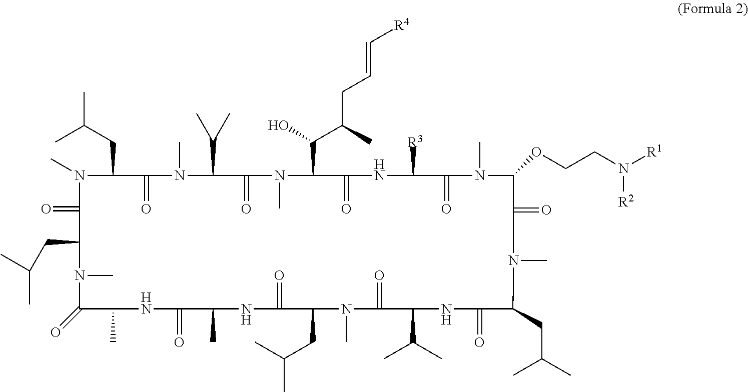

| Compound | R¹ | R² | R³ | R⁴ |
|---|---|---|---|---|
| 2 | —CH₃ | —CH₃ | —CH₂CH₃ | 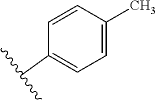 [SMILES: *c1ccc(C)cc1] |
| 3 | —CH₃ | —CH₃ | —CH₂CH₃ | 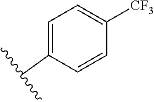 [SMILES: *c1ccc(C(F)(F)F)cc1] |
| 4 | —CH₃ | —CH₃ | —CH₂CH₃ | 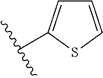 [SMILES: *c1cccs1] |
| 5 | —CH₃ | —CH₃ | —CH₂CH₃ | 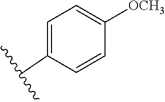 [SMILES: *c1ccc(OC)cc1] |
| 6 | —CH₃ | —CH₃ | —CH₂CH₃ | 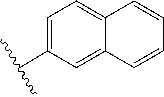 [SMILES: *c1ccc2ccccc2c1] |

In yet another aspect, the present disclosure also relates to a process for preparing the compounds according to Formula 1 and 2. In one embodiment, the compounds may be prepared by a process comprising a compound-forming reaction, wherein the reaction comprises the use of copper triflate, and an amino alcohol. The amino alcohol may be a compound of Formula 3:

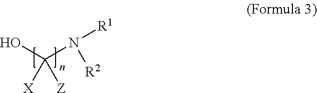

wherein the substituents, X and Z may be independently selected from H, alkyl (e.g. C₁-C₆ alkyl, for example methyl), substituted alkyl, e.g. substituted C₁ to C₆ alkyl or wherein X and Z may be joined together to form a C₃ to C₆ cycloalkyl or heterocycloalkyl ring; and wherein the integer n, R¹, R² are as defined in any one or combination of the embodiments as described herein. In one embodiment, X and Z of Formula 3 are H, and R¹, R² are as defined in any one or combination of the embodiments described herein. In yet another embodiment, X and Z are both H; R¹ and R² are both alkyl, e.g. C₁-C₆ alkyl, for example methyl, and n is 2.

In another aspect, the compounds according to the present disclosure may be prepared or are obtainable by a process comprising reacting a cyclosporin A intermediate comprising a leaving group at the 3 (sarcosine) position, with an amino alcohol compound, such as a compound of Formula 3. In another embodiment, the compounds as described herein may be obtainable according to a process or method comprising: a) reacting a cyclosporin compound, e.g. cyclosporin A with dipyridyl disulphide to form a thiopyridyl cyclosporin intermediate (e.g. [(2′-(2-thiopyridyl)-Sar]³-cyclosporin A) and b) reacting said intermediate with an amino alcohol compound in the presence of copper triflate. The amino alcohols may be compounds as defined according to Formula 3 above. Examples of amino alcohol compounds which may be used include, but are not limited to, morpholino ethanol, or dimethylamino ethanol. The process of preparation of a compound according to the present invention may further comprise a step c) of reacting the compound obtained in step b) with a compound comprising an alkenyl moiety (e.g. a styrene compound; or a vinyl arene or heteroarene compound), and a catalyst, e.g. a Grubbs catalyst.

It is intended that the compounds of present invention may include, in addition to stereocenters as designated or depicted in the formulae, all their enantiomers, diastereomers, racemates or other mixtures, as well as polymorphs, solvates, hydrates, complexes and salts. Unless otherwise indicated, it is also intended that compounds within the scope of the current invention comprising one or more asymmetric centers which are not designated or depicted in the formulae or are named/described herein include also all enantiomers, diastereomers, their mixtures, racemic or otherwise thereof. The representation of double bonds in the current disclosure refer to the isomer as depicted, however optionally, may also include the other Z (or E) isomer. Also included in the context of the present invention is the use of any optically pure or stereochemically pure stereoisomers, as well as any combination of stereoisomers, as determined or prepared by methods well-known in the art.

Optionally, the compounds of the invention may also include their isotopes, such compounds wherein an atom is replaced with an isotope, such as hydrogen with a deuterium, or a carbon with carbon-13.

As defined herein, a pharmaceutically acceptable compound is a compound which is generally safe, non-toxic and neither biologically nor otherwise undesirable, and is acceptable and compatible for pharmaceutical use in humans.

A pharmaceutically acceptable salt is a salt of a compound such as provided herein, which retains its biological properties and which is non-toxic and is compatible for pharmaceutical use.

Salts according to the present disclosure may result from the addition of acids to the compound of Formula 1, Formula 2 or any one of the specific compounds described herein. The resultant acid addition salts may include those formed with acetic, 2,2 dichloroacetic, citric, lactic, mandelic, glycolic, adipic, alginic, aryl sulfonic acids (e.g., benzenesulfonic, naphthalene-2-sulfonic, naphthalene-1,5-disulfonic and p-toluenesulfonic), ascorbic (e.g. L-ascorbic), L-aspartic, benzoic, 4-acetamidobenzoic, butanoic, (+) camphoric, camphor-sulfonic, (+)-(1S)-camphor-10-sulfonic, capric, caproic, caprylic, cinnamic, citric, cyclamic, dodecylsulfuric, ethane-1,2-disulfonic, ethanesulfonic, 2-hydroxyethanesulfonic, formic, fumaric, galactaric, gentisic, glucoheptonic, gluconic (e.g. D-gluconic), glucuronic (e.g. D-glucuronic), glutamic (e.g. L-glutamic), α-oxoglutaric, glycolic, hippuric, hydrobromic, hydrochloric, hydriodic, isethionic, lactic (e.g. (+)-L-lactic and (±)-DL-lactic), lactobionic, maleic, malic (e.g. (−)-L-malic), (±)-DL-mandelic, metaphosphoric, methanesulfonic, 1-hydroxy-2-naphthoic, nicotinic, nitric, oleic, orotic, oxalic, palmitic, pamoic, phosphoric, propionic, L-pyroglutamic, salicylic, 4-amino-salicylic, sebacic, stearic, succinic, sulfuric, tannic, tartaric (e.g. (+)-L-tartaric), thiocyanic, undecylenic and valeric acids. In particular, acid addition salts may include those derived from mineral acids such as hydrochloric, hydrobromic, phosphoric, metaphosphoric, nitric and sulfuric acids; from organic acids, such as tartaric, acetic, citric, malic, lactic, fumaric, benzoic, glycolic, gluconic, succinic, arylsulfonic acids.

The compound of the invention may be useful for the therapy, or prevention of diseases or medical conditions, and in the manufacture of a medicament.

The term ‘therapy’ which may be used synonymously with the term ‘treatment’, as used herein, relates to a therapeutic intervention capable of effecting a cure, improvement, amelioration, control, control of progression, prevention of progression, prevention of reoccurrence of a disease, condition or symptom associated with said disease or condition.

As understood herein the term ‘prevention’, which may be used interchangeably with the term ‘prophylaxis’ refers to the use of a compound, or composition, for preventing the occurrence of a disease, condition or symptom, or significantly reducing the likelihood of occurrence of a disease, condition or symptom, as well as the prevention of, for example, a further reoccurrence of a disease, condition or associated symptom. Also included within the meaning of the term is the prevention of progression of a disease, condition or associated symptom, after an initial improvement or after initial removal of the cause of the disease, condition or symptom.

In particular, the compound according to the invention may be used for the treatment and/or prevention of cyclophilin-mediated disease or condition.

In particular, the compounds as described herein may be used as inhibitors of cyclophilin, especially cyclophilin A (CypA) and/or cyclophilin D (CypD). In one embodiment, the compound is used as an inhibitor of cyclophilin D, for example, provided or administered at a therapeutically relevant amount for the inhibition of cyclophilin D. As generally understood herein, the term ‘therapeutically effective amount’ is an amount which when administered to a subject (e.g. human subject) for treating or preventing a disease or condition, is sufficient to effect such treatment or prevention thereof.

The overexpression of these cyclophilins have been linked or correlated with various diseases and conditions, in particular inflammatory diseases in humans. For example, cyclophilin A has been demonstrated to function as a chemokine to facilitate leukocyte migration in support of an inflammatory response, and the blockade of cyclophilin A has been shown to be beneficial in animal models of acute inflammation. A significant body of evidence now also supports the opening of a pore at the mitochondrial membrane, termed the Mitochondrial Permeability Transition Pore (MPTP), as being critical to the onset and maintenance of a severe form of inflammation, necrotic inflammation. A key regulator of this MPTP opening is Cyclophilin D and inhibition of CypD has shown good activity in preventing tissue damage associated with necrotic inflammation. Opening of the MPTP, and subsequent initiation of necrotic cell death, is triggered by elevated intracellular calcium levels that result from a variety of factors including oxidative stress, hypoxia, bile salt toxins, etc. Pharmacological inhibition of CypD, may therefore be protective toward tissue degradation due to ischemia-reperfusion injury of organ tissue.

It has been found that compounds according to the present disclosure, as evidenced in the Examples, are surprisingly effective as inhibitors of cyclophilin, in particular cyclophilin D. The compounds thus may be useful for the treatment or prevention of disease or conditions, wherein raised levels or activity of cyclophilin is associated with, contributing to, or resulting in said disease or condition. In particular, the cyclophilin-mediated disease or condition which may be treated or prevented according to the invention may be a cyclophilin-D mediated disease or condition. Said disease or condition may, for example, be consequent to mitochondrial dysfunction, for example due to up-regulated opening of the MPTP.

In one embodiment, the compounds according to the present disclosure may be used as a cellular protectant (e.g. for the prevention, or reduction of cell damage or death), or as a mitochondrial protectant (e.g. for the prevention, or reduction of mitochondrial dysfunction or damage).

Cyclophilin-mediated diseases or conditions are typically diseases and conditions associated with inflammatory response, cellular damage, injury and/or cell death (e.g. necrosis) and may include, but are not limited to, the diseases and conditions as further described below.

In one embodiment, a compound according to invention may be used in the treatment and/or prevention of a disease or condition associated with cell injury, or cell death, for example cellular necrosis (unprogrammed cell death, associated with loss of cell membrane integrity and release of cellular components to extracellular matrix). The cell injury and cell death may be a consequence, or induced, for example, injury, infection, infarction, inflammation, ischemia, exposure to toxins, temperature trauma, physical trauma, etc.

As understood herein, the term ‘cell’ or ‘cellular’ may also refer to a collection or aggregate of cells, i.e. cellular tissue. Said tissue may be associated or located in a specific organ such as the kidney, liver, heart, lung, and other organs typically found in the subject to be treated.

In one embodiment, the compounds according to the invention may be used in the treatment or prevention of organ failure, or organ injury. Said organ may be selected from kidney, liver, heart, lung, pancreas, intestine, cornea, skin, brain and nerve tissue. Examples of nerve tissue are for example, central, or peripheral nerve tissue. In one embodiment, the organ is a kidney. As understood herein, organ failure or organ injury may refer to the failure or injury of one, two, or multiple organs in a subject. For example, kidney failure or injury may refer to the condition of one kidney, but optionally also two, or both kidneys of a subject or patient. In one embodiment, the compounds may be used for treating and/or preventing multiple organ failure (for example, kidney, lung, and liver failure).

In another embodiment, the compound according to the invention may be used for the prevention and/or treatment of a disease or condition of the kidney, i.e. a renal disease or condition. A disease or condition of the kidney may be characterized, for example by abnormal or impaired function of a kidney or a kidney tissue. Abnormal or impaired renal function may be determined according to standard clinical diagnostic methods in the art, for example but not limited to, the measurement of renal functional markers such as blood urea nitrogen and/or serum creatinine. In a further embodiment, the compound according to the invention may be used for the prevention and/or treatment of ischemia, i.e. ischemia in a tissue or organ such as described herein. Ischemia, or ischemic injury generally occurs when the blood supply to an area of tissue or is cut-off or interrupted resulting in, amongst other factors, a lack of oxygen or an inadequate supply of oxygen to the tissue. The incidence of ischemic injury may be due to and/or be a result of, for example but not limited, to myocardial infarction, stroke, and other thrombotic events. The length of time a tissue can survive oxygen deprivation varies, but eventually ischemic tissue may become necrotic. In one embodiment, the compound may be used for the treatment of myocardial ischemia, renal ischemia, brain ischemia, or hepatic ischemia.

Ischemic injury may occur during surgery when blood vessels are cross-clamped, and in organs for transplantation. Ischemia-reperfusion (reoxygenation) injury is the tissue damage caused when the blood supply returns to the tissue after a period of ischemia or lack of oxygen (anoxia, hypoxia). Without being bound by theory, it is believed that the absence of oxygen and nutrients from the blood during the ischemic period creates a condition in which the restoration of circulation results in inflammation and oxidative damage.

In an embodiment, the compound according to the present invention may be used in the treatment or prevention of ischemia-reperfusion injury. The ischemia-reperfusion injury may be associated with, or a consequence of a surgical procedure.

The surgical procedure may be a transplantation procedure. The ischemia-reperfusion injury may occur in recipient subjects or in donor subjects. In organ, or organ tissue transplantation, there is a period of time between removing an organ or tissue from the donor's blood supply until the reconnection of the organ or tissue to the donor recipient's blood supply. In some cases, organs may need to be transported long distances to the location of surgery, increasing the likelihood of organ damage. In one embodiment, the invention relates to use of a compound described herein for the treatment or prevention of ischemia-reperfusion injury associate with, or as a consequence of an organ transplantation.

In one embodiment, the compound of the invention is administered to an organ transplant recipient (e.g. a kidney transplant recipient).

In yet a further embodiment, the ischemia-reperfusion injury is renal ischemia-reperfusion injury, which may for instance may arise from surgical procedure where blood vessels supplying a kidney are clamped for duration of at least a portion of the surgical procedure, such as kidney transplantation. In one embodiment, the compound as described herein is used for the prevention or treatment of acute kidney injury.

Kidney transplantation procedures carry risks of conditions such as acute kidney injury, which may be induced or caused by renal ischemia, and renal ischemia-reperfusion injury. Renal ischemia may result from arterial occlusion, shock and kidney transplantation, and can lead to renal cell death and kidney failure. In a further embodiment, the compound as described herein is used for the prevention or treatment of acute kidney injury associated with, or consequent to a kidney transplant procedure. For example, following removal of the donor kidney, the kidney tissue may be subject to oxygen starvation as a result of loss of blood flow (ischemia), and damage to the ischemic renal tissue may further ensue upon re-initiation of flow (reperfusion injury). The prevention of such damage, by administration of a compound which may be used as a protectant by potent inhibition of cyclophilin D and prevention of MPTP opening following ischemic stress may help improve the viability of the transplanted organ.

In other embodiments, the invention may relate to the use of a compound as described herein for the treatment, or prevention of ischemia-reperfusion injury of the liver, or of the heart, optionally consequent to, or associated with transplantation of said organ or organ tissue.

According to the present disclosure, a compound or pharmaceutically acceptable salt thereof as defined herein above may be used, e.g. in the manufacture of a medicament, for preserving an organ and/or protecting an organ from organ injury, such as during transplantation surgery.

In the context of use of the compound, (e.g. in the manufacture of medicament comprising said compound) and in further in the context of transplantation surgery, the compound or medicament may be administered to an organ donor and/or to an organ recipient prior to, during, and/or after transplantation of an organ from said organ donor to said organ recipient.

In one embodiment, the compound of the invention may be administered to the donor subject prior to removal of the donor organ, for example by systemic administration e.g. injection or infusion. Alternatively, or in addition to, a compound according the present invention may be administered to the organ after the removal of the organ from an individual and prior to transplantation or re-attachment. For example, the compound could be added to (or included in) a fluid in which the organ is placed; and/or a compound as described herein could be added to (or included in) a fluid that is recirculated in and or through the organ.

In another embodiment, a compound according to the present invention may be administered to a subject, prior to commencement of surgery, for example to an organ transplant recipient prior to commencement of transplantation surgery. In yet a further embodiment, the compound may be administered during, and/or also after surgery, for example in the case of a transplant recipient during, and/or after transplantation. In yet a further embodiment, the compound may be administered to donor, optionally, the excised organ, and recipient, throughout the duration of the transplantation process and/or recovery periods.

The term ‘donor’ or ‘organ donor’ as used herein refers to a subject from which the organ (or tissue of an organ) will be removed. Said donor may be a live donor. Alternatively, the donor may be a clinically dead donor, the term ‘clinically dead’ as generally understood by the skilled person and defined by standard clinical and/or legal guidelines in the art, for example as applicable to human subjects.

Furthermore, the term ‘subject’ or ‘patient’ may be used interchangeably, and refer in one embodiment to a human subject. Similarly, the term ‘organ donor’ or ‘organ recipient’ or the like as used herein may refer to a human subject. These terms may also refer to other animals, such as other mammals. The invention in further embodiments may also have application, for instance, in farm animals or other veterinary subjects, in particular mammals such cats, dogs, primates, horses, cows, and pigs. The invention may also have application in transgenic animals (e.g. transgenic pigs), where such animals have organs suitable for human transplantation.

A systemic dose of the compound of the invention can be administered to the organ donor prior to organ removal. This allows for the organ to receive a protective dose of the compound prior to removal, thereby preserving the organ by protecting the organ from damage during the removal, and up to and during the process of transplantation into the donor recipient. In the case where more than one organ is being removed from a donor, this systemic dose ensures each organ receives a dose of the compound. A systemic dose is also more likely to provide an even dose of the compound to the organ tissue that is to be transplanted. In the case where the donor is legally dead, the dose can be greater than would normally be given to a living subject.

The compound can be administered shortly before organ removal surgery, or during organ removal surgery. For example, the compound of the invention may be administered up to 8, 7, 6, 5, 4, 3, 2 or 1 hours before surgery.

In addition, or in the alternative, the organ recipient may receive a dose of the compound of the invention directly prior to receiving the organ such that their blood supply contains a protective dose of the compound of the invention, thereby preserving the transplanted organ, or body part from damage after surgery.

In the embodiments of the compound (or method or use) as mentioned herein above, the organ may be any transplantable organ and can be, for example, a kidney, liver, heart, lung, pancreas, intestine, cornea, skin, brain and nerve tissue.

As described above, the present disclosure provides for administration of the compounds, acting as cyclophilin inhibitors to prevent, treat, ameliorate and/or reduce damage to organs. Optionally, the treatment may also be applied to treatment and prevention of damage to body parts such as limbs, hands, feet, fingers or toes. For example, in accidents involving severed limbs, there is a period of time between the severing of the body part from the blood supply until the reconnection of the body part to the blood supply. During this period there may also be the potential for ischaemia-reperfusion injury. In some cases, body part and patients may need to be transported long distances to the location of surgery, increasing the likelihood of damage before, during and after re-attachment. In case of body parts, these may be severed from and re-attached to the same individual, or may be given to a second individual as a transplant. There the body part is severed from a subject, the severing may be complete or partial. Partial severing may be for example severing of the blood supply but the body part remaining attached for example via skin, bone or muscle tissue. The compound may administered to (i) to a severed body part; and/or (ii) to the subject prior to re-attachment of the body part; and/or (iii) to the subject during or after re-attachment of the body part.

The compound of the invention may optionally be administered together with one or more further active substances.

In a further aspect, the present disclosure may also provide for a compound described herein (i.e. a compound of Formula 1, or Formula 2 or specific embodiments thereof) for use in the treatment or prevention of a kidney condition or disease in a subject exposed to a nephrotoxin capable of inducing said kidney condition or disease, wherein the nephrotoxin is a nephrotoxic drug substance or an endogenous nephrotoxin.

Nephrotoxins are compounds or substances which are capable of disrupting or impairing the function of a subject's kidney(s) and its associated tissues. In one embodiment of the disclosure, the nephrotoxin capable of inducing a kidney condition or disease is a nephrotoxic drug substance.

As used herein, the term ‘nephrotoxic drug substance’ is an active pharmaceutical ingredient, or a pharmacologically- or diagnostically-active compound or mixture of compounds useful for medical or therapeutic applications in the prevention, diagnosis, stabilization, treatment or management of a condition, disorder or disease and which is capable of disrupting, impairing, or reducing renal function. A nephrotoxic drug substance may be provided or administered to a subject as a medicament or pharmaceutical dosage form comprising said nephrotoxic drug substance or a mixture of nephrotoxic substances, and one or more non-pharmacologically active excipients or carriers. In particular, a nephrotoxic drug substance may be a dose-limited drug substance where administration for its indicated therapeutic or diagnostic applications is restricted in terms of a threshold dose amount given at a single and/or cumulative dose due to its potential for nephrotoxic side effects. A nephrotoxic drug substance may also be further defined as a drug substance for which nephrotoxicity is listed as a side effect or as an adverse effect as per its prescribing information, and/or where its prescribed use for its intended therapeutic/diagnostic application includes an advisory for the monitoring of the dose concentration of the nephrotoxic drug substance (e.g. its serum concentration) in a subject to which it is administered, and/or the renal function of the recipient subject, for example, for signs and markers associated with nephrotoxicity.

As understood herein, the phrase ‘exposure to a nephrotoxin’ or similar, may refer to the exposure of a subject to a nephrotoxin during the course of a treatment for a condition, symptom or disease, wherein a subject is administered for therapeutic or diagnostic purposes, one or more doses of a nephrotoxin such as any one or combination of the nephrotoxic drug substances as defined in various embodiments herein. The phrase ‘exposure to a nephrotoxin’ as used herein also comprises any unintended exposure of a subject to a nephrotoxin, for example, but not limited to, accidental exposure such as from a needle-stick injury, or situational/unanticipated circumstances such as physical trauma or prolonged physical stress which may cause the release and/or build-up of endogenous nephrotoxins.

As understood herein, the term ‘drug substance’, as well as its genus, family, or species may refer to the drug substance as such, as well as any pharmaceutically acceptable salt, hydrate, derivative, or prodrug thereof. For example, the term ‘gentamicin’ may include also its common commercially available form, gentamicin sulfate. Similarly, the term ‘aminoglycosides’, interchangeable with the term ‘aminoglycoside antibiotics’ for example refers to any compound falling within its common definition or classification in the art.

In one particular embodiment, the nephrotoxic drug substance is a chemotherapeutic agent. The chemotherapeutic agent is preferably a cytotoxic or antineoplastic agent used in the treatment cancer in a subject, for example for the targeting and killing of tumour cells. In one specific embodiment, the chemotherapeutic agent is selected from the group consisting of platins (e.g. carboplatin, cisplatin, oxaliplatin or nedaplatin), anthracyclines (e.g. daunorubicin, doxorubicin, idarubicin, epirubicin), bleomycin, mitomycins, actinomycins, cyclophosphamides, capecitabine, cytarabine, gemcitabine, ifosfamide, interleukin-2, streptozocin, gemtuzumab ozogamicin, melphalan, methotrexate, pemetrexed, plicamycin, and trimetrexate.

Preferably, the chemotherapeutic agent is cisplatin. Cisplatin, a platinum complex, is used to treat a variety of cancers including ovarian, lung, head, neck, testicular, and bladder cancers. However, high doses are restricted as cisplatin may induce cumulative and dose-dependent nephrotoxicity. Cisplatin is taken up by renal tubular cells, especially the proximal tubular cells of the inner cortex and outer medulla.

These cells and sites are subject to injury and necrotic cell loss, resulting in acute kidney injury and impairment of renal function. Renal toxicity becomes more severe with repeated courses of treatment with cisplatin, and methods for reduction of nephrotoxicity of cisplatin include the use of a 6 to 8 hour infusion with intravenous hydration. In a specific embodiment of the present disclosure, Compound of Formula 1 is used for the treatment and/or prevention of cisplatin-induced acute-kidney injury.

In one embodiment, a compound of Formula 1 (or Formula 2) or a pharmaceutically acceptable salt thereof is administered to a subject undergoing cancer treatment, wherein the cancer treatment comprises administration of a chemotherapeutic agent to the subject. In one embodiment, the subject may be administered a dose of a compound according to the present disclosure prior to receipt of a dose of the chemotherapeutic agent, for example a dose of cisplatin. The compound of Formula 1 (or Formula 2) may be also administered throughout a treatment course of chemotherapy, or may be administered to a subject undergoing cancer treatment with a chemotherapeutic agent, said subject having developed a kidney condition or disease subsequent to, or as a result of exposure to said chemotherapeutic drug. Said chemotherapeutic drug may be cisplatin.

In another embodiment of the present disclosure, the nephrotoxic drug substance may be an antimicrobial agent. Preferably, the antimicrobial agent is an antibiotic agent which is active against bacteria, for example gram-negative and/or gram-positive bacteria). In one embodiment, the antimicrobial agent may be selected from a group consisting of aminoglycosides, beta-lactams, polypeptide antibiotics, glycopeptide antibiotics peptidomimetic antibiotics, outer membrane protein targeting antibiotics and antifungal agent (e.g. amphotericin B) and combinations thereof.

In one specific embodiment, a compound of Formula 1, or a compound of Formula 2 or a pharmaceutically acceptable salt thereof is used for the prevention or treatment of a kidney condition or disease induced by exposure to a nephrotoxic drug substance, wherein the drug substance is an aminoglycoside antibiotic. Aminoglycoside antibiotics are used in particular for clinical management and treatment bacterial infections, in particular gram-negative bacterial infections. Aminoglycosides have been found to contribute to renal tubule cells injury and necrosis. A compound as described herein may be used, for the protection of renal function in subjects receiving aminoglycoside antibiotic treatment. In one embodiment, the aminoglycoside antibiotic is selected from the group consisting of gentamicin, tobramycin, amikacin, netilmicin, apramicin, streptomycin, kanamycin, neomycin and sisomycin. In a preferred embodiment, the aminoglycoside antibiotic is gentamicin. Gentamicin is administered as an injection (intramuscular or intravenously) for the treatment of serious infections caused Staphylococcus species, Citrobacter species, Enterobacter species, Escherichia coli, Klebsiella-Enterobacter-Serratia species, Proteus species and Pseudomonas aeruginosa. While effective as an antibiotic, its use requires careful monitoring and control by a clinician, due to its potential for adverse renal effects and nephrotoxicity, which is characterized by rise in renal function markers such as blood urea nitrogen (BUN), serum creatinine, or oliguria. Dosing of gentamicin needs to be carefully monitored, and adjusted and closely monitored, especially for patients with impaired renal function in order to maintain a therapeutically relevant but not excessive levels of the drug.

In one embodiment, the compounds according to the present invention may be used as protectants against reduction in kidney function, in particular gentamicin-induced kidney conditions, i.e. acute kidney injury. In one particular embodiment of the present disclosure, a compound of Formula 1 or Formula 2 or a pharmaceutically acceptable salt thereof may be used for the treatment and/or prevention of gentamicin-induced acute kidney injury. The compound or a medicament comprising said compound may be used for the treatment of a subject suffering from an infection requiring treatment with gentamicin, wherein said subject is administered gentamicin. In one embodiment, a dose of a compound according to the present disclosure may be administered to a subject prior to administration of a dose of gentamicin. In another embodiment, said compound may be administered to a subject after the onset of reduced renal function, for example subsequent to exposure to gentamicin.

In a further embodiment, the antimicrobial agent is a beta lactam. Examples of beta-lactam antibiotics include but are not limited to cephalosporins, and penicillins, including ureidopenicillins (e.g. piperacillin), aminopenicillins, carboxypenicillins, carbapenems. Also include within the context of the present disclosure, are combination treatments with beta-lactamase inhibitors such as tazobactam, sulbactam, and clavulanic acid.

In another embodiment, the antimicrobial agent is a polypeptide, glycopeptide or peptidomimetic antibiotic. Examples of a polypeptide antibiotics which may comprise of non-ribosomal polypeptides, include bacitracin, and polymyxins such as polymyxin A, B, C, D, E (colistin). Examples of glycopeptide peptides are vancomycin, teicoplanin.

The antimicrobial agent may be based on a naturally derived peptide or glycopeptide or alternatively may be a synthetic or semi-synthetic e.g. a peptidomimetic compound, with amino acid modifications. An example of a peptidomimetic antibiotic is murepavadin, an outer membrane protein-targeting antibiotic for use in the treatment of serious infections associated with Pseudomonas aeruginosa.

In one embodiment, the antimicrobial agent is murepavadin. In one embodiment, a compound of Formula 1, or of Formula 2 or a pharmaceutically acceptable salt thereof as described herein is administered to a subject suffering from an infection (e.g. a Pseudomonas aeruginosa infection), wherein said infection is treated by the administration of murepavadin. In said embodiment, a dose of compound of Formula 1, or of Formula 2 or a pharmaceutically acceptable salt thereof as described herein may be administered prior to the administration of a dose of murepavadin. compound of Formula 1, or of Formula 2 or a pharmaceutically acceptable salt thereof may be administered also throughout the course of prescribed treatment with murepavadin.

In another embodiment, a compound of Formula 1, or of Formula 2 or a pharmaceutically acceptable salt thereof may be administered to a subject suffering from an infection (e.g. a bacterial and/or fungal infection), wherein said infection is treated by administering the antimicrobial agent to the subject. In one embodiment, the subject may be administered a dose of the compound prior to receipt of a dose of the anti-microbial agent, for example prior to receiving a dose of gentamicin. The compound may be also administered throughout a treatment course of the antimicrobial agent, or may be administered to a subject undergoing treatment with said antimicrobial agent, who has developed a kidney condition or disease subsequent or as a result of exposure to said antimicrobial agent. In said embodiments, preferably the antimicrobial agent is an aminoglycoside antibiotic such as gentamicin.

In another embodiment, the antimicrobial agent is an anti-fungal agent active against fungal species such as but not limited to Aspergillus, Candidia, Cryptococcus, and used for treatment of subjects suffering from fungal infections. Examples of anti-fungal agents include 5-fluorocytosine, amphotericin B, fluconazole, and caspofungin.

In yet another embodiment, the nephrotoxic drug substance is a blood pressure controlling medication or medicine, such as an ACE (angiotensin-converting-enzyme) inhibitor, or an angiotensin receptor blocker. A compound of Formula 1, or of Formula 2 or a pharmaceutically acceptable salt thereof, as described herein may be administered to a subject suffering from high blood pressure or a condition requiring reduction of blood pressure, wherein said conditions are treated by administration of said blood pressure controlling medication or agent to the subject. Examples of ACE inhibitors include, but are not limited to captopril, ramipril, benazepril, enalapril, fosinopril, lisonopril, quinapril. Examples of angiotensin receptor blockers include, but are not limited to candesartan, valsartan, telmisartan, irbesartan, olmesartan, telmisartan, eprosartan, and losartan.

In another embodiment, the nephrotoxic drug substance is a macrolactone immunosuppressive agent. These compounds, which are also referred to as macrolide immunosuppressants, may be used in the prevention or treatment of conditions or diseases such as organ transplant rejection, in an organ transplant recipient. In one embodiment, the macrolactone immunosuppressive agent is tacrolimus, or an mTor inhibitor such as sirolimus (rapamycin).

In another embodiment, the nephrotoxic drug substance is an HIV protease inhibitor. a compound of Formula 1, or of Formula 2 or a pharmaceutically acceptable salt thereof may be administered to a subject diagnosed or suffering from HIV or a related condition, wherein said HIV or related condition is treated by administering the HIV protease inhibitor to the subject. Examples of HIV protease inhibitor include, but are not limited to indinavir and ritonavir.

In one embodiment, the nephrotoxic drug substance is a peptic ulcer drug or medication. A compound of Formula 1, or of Formula 2 or a pharmaceutically acceptable salt thereof may be administered to a subject diagnosed or suffering from an ulcer e.g. a stomach ulcer, wherein ulcer is treated by administering the peptic ulcer drug or medication to the subject. Examples of peptic ulcer drugs include, but are not limited to, cimetidine, esomeprazole, lansoprazole, omeprazole, pantoprazole, and rabeprazole.

In one embodiment, the nephrotoxic drug substance is a non-steroidal anti-inflammatory drug (NSAID). A compound of Formula 1, or of Formula 2 or a pharmaceutically acceptable salt thereof may be administered to a subject suffering from pain, fever and/or inflammation, wherein said pain, fever and/or inflammation is treated by administering the non-steroidal anti-inflammatory drug (NSAID) to the subject. Alternatively, the compound according to the present disclosure may also be administered to a subject having taken an overdose of an NSAID. Examples of NSAIDs, include but are not limited to ibuprofen, ketoprofen, diclofenac and aspirin.

In one embodiment, the nephrotoxic drug substance is a laxative. A compound of Formula 1, or of Formula 2 or a pharmaceutically acceptable salt thereof may be administered to a subject suffering from constipation, wherein said constipation is treated by administering the laxative to the subject. Alternatively, a compound according to the present disclosure may also be administered to a subject having taken an overdose of a laxative. An example of a laxative in the context of the present disclosure is sodium phosphate.

In yet another embodiment, the nephrotoxic drug substance is a contrast agent. A contrast agent is a substance used as a diagnostic tool for the visualization of internal organs or tissues. The contrast agent may be administered intravenously. In one specific embodiment, the contrast agent is an iodinated contrast agent, for example, but not limited to diatrizoate, iothalamate, iohexol, iodixanol, or iopamidol. A compound of Formula 1, or of Formula 2 or a pharmaceutically acceptable salt thereof may be used in the prevention or treatment of contrast-agent induced acute kidney injury or nephropathy, and may be administered to a subject undergoing, having undergone, or requiring diagnosis using a contrast agent, such as an iodinated contrast agent.

The subject to be treated with or administered with a compound according to the present disclosure, or a pharmaceutically acceptable salt thereof, may, in one embodiment, be administered a nephrotoxic drug as a therapeutic remedy or for an in vivo diagnostic application. In other embodiments, the subject may receive more than one nephrotoxic drug substance. For example, the subject treated or administered with a compound of Formula 1, or Formula 2 or a pharmaceutically acceptable salt thereof may be concomitantly receiving more than one drug substance, for example as a combination treatment, or alternatively, as separate treatment for different conditions or different aspects or symptoms relating to a condition or disease. In one embodiment, the subject to which a compound according to the present disclosure is administered may be undergoing a course of treatment with a specific combination of a nephrotoxic drug substance, and a second further drug substance (e.g. piperacillin/tazobactam) optionally wherein the second further drug substance is also a nephrotoxic drug substance. In one embodiment, treatment with one nephrotoxic drug may predispose or enhance the risk of nephrotoxicity of another (nephrotoxic) drug substance. In other embodiments, the subject may be receiving more than one drug substance during a time period in which a compound of the present disclosure is administered.

In another embodiment of the disclosure, the nephrotoxin capable of inducing a kidney condition or disease is an endogenous nephrotoxin. As defined herein, an endogenous nephrotoxin is a molecule or substance (e.g. a protein) produced endogenously by a subject and is not externally administered, in contrast to the nephrotoxic drug substances described above, which may be considered as exogenous toxins. The endogenous nephrotoxin may be present in the subject or a subject's blood or blood serum at a non-nephrotoxic concentration or amount during normal physiological and homeostatic conditions, however at elevated levels i.e. above a threshold or baseline concentration may become nephrotoxic, degrade or breakdown to nephrotoxic components and/or trigger cellular or inflammatory response events leading to onset of nephrotoxicity, and kidney tissue injuries.

In one embodiment, the endogenous nephrotoxin is myoglobin, and optionally any breakdown or degradation products or released components associated with myoglobin. Myoglobin is an oxygen and iron binding protein found in muscle tissue. High levels of myoglobin and its related components may be directly toxic to kidney tubular cells, and may also lead to renal vasoconstriction, formation of intratubular casts amongst other pathologies.

Rhabdomyolysis is a condition characterized by injury or breakdown of skeletal muscle tissue, wherein their contents are released into circulation. The release of high levels of myoglobin is also associated with the related condition of myoglobinuria. Other conditions where endogenous cellular components may become nephrotoxic include, but are not limited to, conditions such as hemolysis (red blood cell lysis, where contents of damaged red blood cells e.g. heme are released into circulation), and also tumour lysis, or myeloma. Tumours in cancer patients may lyse (for example in the course of chemotherapy) and release tumour cellular content into circulation and to the kidneys.

In particular, a subject may suffer from, or may be at risk to suffer from rhabdomyolysis, and its related conditions and be at risk for exposure to endogenous nephrotoxins such as myoglobin due to a number of factors, in particular physical activity or trauma. In one embodiment, a compound of Formula 1, or of Formula 2 or a pharmaceutically acceptable salt thereof is used for the prevention and/or treatment of a kidney condition or disease induced by exposure to an endogenous nephrotoxin (e.g. rhabdomyolysis or myoglobinuria-induced acute kidney injury), and is administered to a subject having experienced, or is suffering from any one or combination of: physical trauma, crush injury, extreme physical exertion or activities, temperature extremes, exposure to electrical current, and other activities or events which may lead to muscle tissue damage and the breakdown of muscle fibres and/or blood cells.

In a further and related embodiment, a compound according to the present disclosure, or a pharmaceutically acceptable salt thereof, may be administered to the subject prior to exposure to, or engagement with activities (e.g. extreme physical activity or exertion) associated with, or at risk for onset of rhabdomyolysis. Extreme or physical activity or exertion may for example be strenuous exercise which causes or results in skeletal muscle injury as well as optionally severe dehydration.

Subjects which may be at risk for onset of rhabdomyolysis includes subjects exposed to toxins, or drug substances, such as statins which may potentially cause or lead to myopathy. Optionally, a compound of Formula 1 (or Formula 2) or a pharmaceutically acceptable salt thereof may also be used for the prevention or treatment of rhabdomyolysis and its associated conditions or diseases in a subject with an inherited myopathy.

A compound of Formula 1 (or Formula 2), or a pharmaceutically acceptable salt thereof is provided, in another embodiment, for use in the prevention and/or treatment of rhabdomyolysis hemolysis, myoglobinuria, or optionally tumour lysis or myeloma-induced acute kidney injury, or failure.

In one embodiment, the subject to which a compound according to the present disclosure or a pharmaceutically acceptable salt thereof may be administered may have elevated serum and/or urine myoglobin levels, i.e. elevated concentration of myoglobin in blood serum and/or in urine. Alternatively, or in addition, said subject may also have any one or combination of elevated serum levels, i.e. elevated serum concentrations of: creatine phosphokinase, lactate dehydrogenase, calcium, potassium, phosphates; indicating the presence of muscle damage. In a further related embodiment, the subject to which a compound of Formula 1, or Formula 2 or a pharmaceutically acceptable salt thereof is provided has a serum creatine phosphokinase level of at least 5 times higher than baseline.

As defined herein, the term ‘baseline’ used in connection with serum concentration levels of creatine phosphokinase refers to a clinically applicable or expected serum creatine phosphokinase level or range for an individual not yet been exposed to a nephrotoxin, or nephrotoxic levels or concentrations of an endogenous nephrotoxin e.g. myoglobin, factoring in variability which may be due to any one or combination of criteria such as, but not limited to, age-group, gender, existing co-morbidities and the like. The baseline value or baseline range for serum creatine phosphokinase, or any of the other markers may be within the knowledge of the skilled clinician or may be determined based on common methods of the art.

In one embodiment, the nephrotoxic drug substance is administered repeatedly to a subject. In other words, the drug substance is administered more than once, i.e. at least twice. The nephrotoxic drug substance may be administered at regular intervals over a period of time, such as over the course of a clinically-determined treatment period. In some embodiments, the nephrotoxic drug substance may be administered at least once daily over a period of at least 3 days, or at least 7 days, or may be administered at least once daily between 7 and 10 days. In an alternative embodiment, the nephrotoxic drug substance may be administered once, for example for a diagnostic use.

A compound of Formula 1, or of Formula 2 or a pharmaceutically acceptable salt thereof as described herein may be used for the prevention and/or treatment of a kidney condition or disease in a subject induced by a nephrotoxic drug substance, or an endogenous nephrotoxin. In a preferred embodiment, kidney condition or disease is a nephrotoxin-induced acute kidney injury or kidney failure. In one embodiment, the induced acute kidney injury is a prerenal acute kidney injury, for example associated with a reduced blood flow to the kidneys. For example, ACE inhibitors and angiotensin receptor blockers can impair renal perfusion. NSAIDs may also decrease glomerula filtration rate. In further embodiment, the induced acute kidney injury is intrinsic, with damage to cellular or tissues to the kidney, including to the glomerulus, tubules (acute tubular injury or necrosis), intersititum and/or vasculature. Acute tubular injury or necrosis may occur, for example due to accumulation or localization of a cytotoxic drug substance to the tubular cells.

In one embodiment, a compound as described herein e.g. according to Formula 1, or according to Formula 2 or a pharmaceutically acceptable salt thereof may be administered to a subject having a pre-existing condition or disease, which increases the subject's risk of developing a kidney condition or disease when exposed to a nephrotoxin as defined herein. For example, the subject may have an existing condition or comorbid disease such as existing dysfunction or impairment in an organ such as the lung (e.g. chronic obstructive pulmonary obstruction), liver (e.g. a chronic liver disease) or heart (for example coronary artery disease, or heart failure), and/or may have recently undergone major surgery associated with said organ. In a further embodiment, the subject is geriatric (of advanced age) and/or has diabetes.

In a further embodiment, the subject may also have already existing condition of the kidney, for example chronic kidney disease; polycystic kidney disease, kidney stones, or kidney inflammation. Optionally, the subject has a history of renal impairment, and/or requires dialysis.

In yet a further embodiment, a compound or a pharmaceutically acceptable salt thereof as described herein may be administered to a subject with reduced renal function. Reduced renal function may be characterized by any one or combination of: blood urea nitrogen (BUN) levels at least 1.5 to 3 times higher than baseline, and/or serum creatinine levels at least 1.5 to 3 times higher than about baseline, and/or oliguria. In one embodiment, the subject has reduced renal function characterized by serum creatinine and BUN levels at least 2 times higher than baseline.

As defined herein, the term ‘baseline’ used in connection with serum levels, i.e. serum concentration of creatinine and/or blood urea nitrogen (BUN) levels, i.e. blood urea nitrogen concentration, may refer to the baseline values of these renal function markers which were determined for a subject prior to commencement of exposure to a nephrotoxic drug substance (e.g. prior to a treatment regimen comprising administration of a nephrotoxic drug substance). In circumstances where the subject has not had serum creatinine or BUN levels measured prior to exposure to the nephrotoxin and prior to the onset of reduced renal functions, the term ‘baseline’ may refer to the clinically applicable or expected serum creatinine and/or blood urea nitrogen values, or range of values for an individual not yet exposed to the nephrotoxin, factoring variability which may be due to criteria such as, and not limited to, age-group, gender, existing co-morbidities and the like. These baseline values or range of values may be within the knowledge of the skilled clinician, and/or may be determined within common methods of the art.

A compound of Formula 1, or Formula 2, or a pharmaceutically acceptable salt thereof may be administered, in one embodiment, to a subject prior the subject's exposure to a nephrotoxic drug substance. Administration of a compound of Formula 1 or Formula 2 prior to exposure, as understood herein refers to administration of a first dose of said compound before a first dose of a nephrotoxic drug is administered.

Additionally, a dose of a compound according to the present disclosure, or a pharmaceutically acceptable salt thereof, in some embodiments, may be administered before any dose of the nephrotoxic drug substance is administered, such as if a nephrotoxic drug substance is administered repeatedly e.g. more than once during its prescribed course of treatment. Doses of the compound may thus be administered in a period between successive doses of the nephrotoxic drug substance. Optionally, more than one dose of a compound according to the present disclosure may be administered in a period between successive doses of a nephrotoxic drug substance.

In one embodiment, the present disclosure provides for a compound according to the present disclosure or a pharmaceutically acceptable salt thereof for use in the prevention or treatment of a kidney condition or disease induced by a nephrotoxic drug substance (e.g. acute kidney injury) as defined herein, wherein the compound or a pharmaceutically acceptable salt thereof is administered repeatedly to a subject during a first period of time which commences before and overlaps with a second period of time wherein the subject is exposed to, or is administered repeatedly the nephrotoxic drug substance. As used herein, ‘repeatedly’ refers to administration or exposure at least twice, i.e. more than once. The period of time may be understood as a course or period of treatment which is clinically determined for example as therapeutically relevant in regards to intended pharmacological effect in prevention, stabilization, treatment or management of the condition, disorder or disease.

As used herein, the term ‘dose’ or ‘dosage’ as such refers to a single, or unit dose of a compound as described herein or a pharmaceutically acceptable salt thereof, or a drug substance, unless prefaced or followed by an indication of time, time interval or indication of quantity. A ‘daily dose’ or ‘dosage per day’ for example refers to the total dose amount of a compound as described herein, or drug substance administered in the course of one day (24 hours). A daily dose may comprise only one dose, if only one dose is administered once per day but may also be a total based on the sum of multiple unit doses that administered during a day, for example, if more than one unit dose is administered at two or more timed intervals during a day. Intervals between doses may be, for example, two doses administered approximately every 12 hours, or three doses administered approximately every 8 hours. Also as used herein a dose of a compound may refer to a unit dose of Compound of Formula 1, or a pharmaceutically acceptable salt thereof, but may also be applicable to a medicament, or composition or dosage form comprising said unit dose of compound or a pharmaceutically acceptable salt thereof.

In one embodiment, a dose of a compound according to the present disclosure or a pharmaceutically acceptable salt thereof may be administered to a subject within 24 hours or less, before a dose of the nephrotoxic drug substance is administered to the subject. In another embodiment, the compound or a pharmaceutically acceptable salt thereof may be administered to a subject within 24 hours or less, before a dose of an aminoglycoside, e.g. gentamicin is administered to the subject.

In one embodiment, a compound as described herein e.g. of Formula 1, or Formula 2 or a pharmaceutically acceptable salt thereof is administered to the subject after the onset of reduced renal functions. The onset of reduced renal functions may be characterized by, amongst other physiological markers, elevated levels of serum creatinine and/or blood urea nitrogen (BUN) and/or oliguria. In one embodiment, said onset of reduced renal function may be characterized by blood urea nitrogen levels of at least 1.5 to 3 times higher than baseline and/or serum creatinine levels of at least 1.5 to 3 times higher than baseline and/or oliguria. In one embodiment, the subject has reduced renal function characterized by serum creatinine and BUN levels at least 2 times higher than baseline.

The onset of reduced renal function may in one embodiment be due to exposure to a nephrotoxic drug substance. For example, a subject may during the course of the treatment with a nephrotoxic drug substance suddenly develop renal impairment or dysfunction due to accumulation (e.g. blood concentration or localization to specific kidney cells or tissue) of a nephrotoxic drug substance, i.e. exposure to a cumulative dose of the nephrotoxic drug substance. Co-morbidities to the disease or condition arising or worsening during the course of treatment with a nephrotoxic drug substance may contribute to an onset of reduced renal function, leading to acute kidney injury. In other embodiments, the onset of reduced renal function may be due to exposure to an endogenous nephrotoxin, for example as describe in any one of the above embodiments and under any of the above described conditions leading to build-up of endogenous nephrotoxin.

When used herein the term ‘about’ or the like in connection with an attribute or value such as dose amount includes the exact attribute or precise value, as well as any attribute or value typically considered to fall within the normal or accepted variability associated with the technical field, and methods of measuring or determining said attribute or value. The term allows for any variation which in the common practice would allow for the product being evaluated to be considered bioequivalent in a mammal to the recited strength or dose of a claimed product.

In a further aspect, the present disclosure provides for a compound or a pharmaceutically acceptable salt as described herein thereof for use in the prevention and/or reduction of blood urea nitrogen levels of at least 1.5 to 3 times higher than baseline and/or serum creatinine levels of at least 1.5 to 3 times higher than baseline in a subject exposed to a nephrotoxin, e.g. a nephrotoxic drug substance or endogenous nephrotoxin, such as defined in any one of the above embodiments or combinations of embodiments.

In a further embodiment, a compound e.g. of Formula 1, or of Formula 2 or a pharmaceutically acceptable salt thereof may be used for the prevention, or for the reduction of blood urea nitrogen and serum creatinine levels at least 2 times higher than baseline in a subject exposed to a nephrotoxin, wherein the nephrotoxin is a nephrotoxic drug substance (e.g. gentamicin, or any one or combination of substances described herein). In yet a further embodiment, Compound of Formula 1, Formula 2 or a pharmaceutically acceptable salt thereof may be used for the prevention and/or reduction of reduction of blood urea nitrogen and serum creatinine levels at least 1.5 to 3 times higher than baseline in a subject exposed to a nephrotoxin, wherein the nephrotoxin is an aminoglycoside antibiotic, preferably gentamicin.

In another embodiment, Compound of Formula 1 or Formula 2, or a pharmaceutically acceptable salt thereof is used for the reduction of blood urea nitrogen and/or serum creatinine levels in a subject, for example a subject exposed to a nephrotoxic drug substance (e.g. gentamicin) or an endogenous nephrotoxin, optionally wherein the blood urea nitrogen and/or serum creatinine level is reduced after administration of a dose, e.g. a first dose of compound of Formula 1 or a compound of Formula 2. The reduction of BUN and serum creatinine may be determined by comparing BUN and serum creatinine values measured, using methods established in the art, prior to, and after administration of a (e.g. first) dose of compound of Formula 1, or a compound of Formula 2.

It is to be understood, that the use of a compound of Formula 1, or a compound of Formula 2, or a pharmaceutically acceptable salt thereof, or their use in a method of treatment or prevention as described in any one of the embodiments or combination of embodiments described herein may also provide for the manufacture or preparation of a medicament or medicine adapted and prescribed for said uses or methods of treatment and/or prophylaxis.

A compound or a pharmaceutically acceptable salt thereof according to the present disclosure may be administered enterally or parenterally to a subject. In one embodiment, the compound, or a composition or a medicament comprising said compound of Formula 1, or of Formula 2 or a salt thereof may be adapted for administration or may be administered parenterally, for example by intravenous injection or by sub-cutaneous, or intramuscular injection, or by intravenous or subcutaneous infusion. In an alternative embodiment, the compound, or a composition or medicament comprising the compound may be adapted for administration, or may be administered to a subject enterally, for example orally.

The present invention may also relate to a medicament, or a pharmaceutical composition comprising a compound according to any one or combination of the embodiments described herein above, e.g. a compound of Formula 1, or of Formula 2 or a pharmaceutically acceptable salt thereof and one or more pharmaceutically acceptable excipients. The medicament or composition may comprise a therapeutically effective amount, or unit dose(s), of said compound. The medicament, or pharmaceutical composition comprising said compound may be formulated in a dosage form suitable or adapted for injection or infusion by any of the administration methods above. Alternatively, for oral administration, the medicament or pharmaceutical composition comprising a compound according to the present disclosure may be provided in a dosage form suitable or adapted for oral administration, for example such as, but not limited to a tablet, capsule, gelcap, or film. Said medicament, or pharmaceutical composition may be used in accordance with any of the methods of treatment or prevention, or uses described herein.

The following list of numbered items are embodiments comprised in the present disclosure:
1. A compound of Formula 1, or a pharmaceutically acceptable salt thereof,

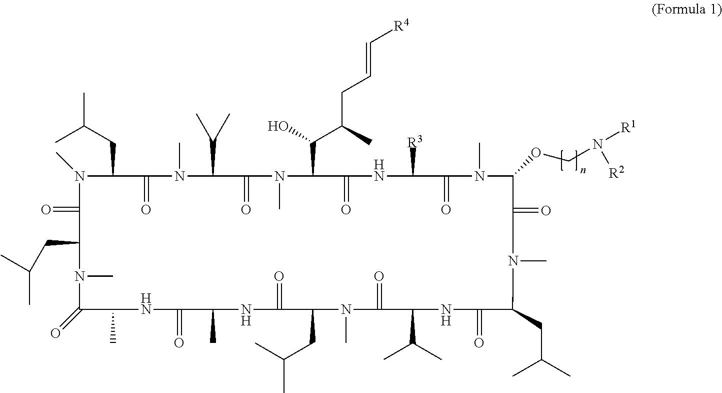

wherein:
n is selected from an integer between 2 and 5;
R¹ and R² are independently selected from H, C₁ to C₆ alkyl or wherein R¹ and
R² may be joined together to form a C₃ to C₆ cycloalkyl or heterocycloalkyl ring;
R³ is ethyl, 1-hydroxyethyl, isopropyl, or n-propyl; and
wherein R⁴ is aryl, substituted aryl, heteroaryl and substituted heteroaryl, and
wherein the substitution is optionally one or more substituents independently selected from C₁ to C₆ alkyl, halogen, haloalkyl, hydroxyl, C₁ to C₆ alkoxyl, amino, monoalkylamino, dialkylamino, thioalkyl, nitro, cyano, carboxyl, alkoxycarbonyl, aryl and heteroaryl.
2. The compound according to item 1, wherein at least one of R¹ or R² is C₁ to C₆ alkyl.
3. The compound according to item 2 wherein R¹ and R² are both —CH₃.
4. The compound according to item 1, wherein R¹ and R² are joined together to form a C₃ to C₆ cycloalkyl or heterocycloalkyl ring.
5. The compound according to item 4 wherein R¹ and R² are joined together to form a morpholinyl residue.
6. The compound according to any one of the preceding items, wherein n is 2.
7. The compound according to any one of the preceding items, wherein R³ is ethyl; or
   wherein R³ is ethyl, n is 2, and R¹ and R² are both —CH₃ (methyl).
8. The compound according to any one of the preceding items, wherein R³ is isopropyl, n-propyl, or 1-hydroxyethyl; or
   wherein R³ is isopropyl, n-propyl, or 1-hydroxyethyl, n is 2, and R¹ and R² are both —CH₃ (methyl).
9. The compound according to any one of the preceding items, wherein R⁴ is aryl or substituted aryl.
10. The compound according to any one of the preceding items, wherein R⁴ is phenyl, or substituted phenyl, wherein the substitution is optionally one or more substituents independently selected from C₁ to C₆ alkyl (e.g. methyl, or t-butyl), halogen (e.g. chloro, fluoro, bromo, or iodo), haloalkyl (e.g. trifluoromethyl), hydroxyl, C₁ to C₆ alkoxyl (e.g. methoxy, phenoxy, or t-butoxy), amino, monoalkylamino, dialkylamino (e.g. dimethylamino), thioalkyl, nitro, cyano, carboxyl ((e.g. —COOCH₃), alkoxycarbonyl (e.g. acetoxy), aryl (e.g. phenyl) and heteroaryl.
11. The compound according to any one of items 1 to 9, wherein R⁴ is naphthalene, or substituted naphthalene.
12. The compound according to any one of items 1 to 8, wherein R⁴ is heteroaryl or substituted heteroaryl.
13. The compound according to item 12, wherein the R⁴ is selected from the group consisting of pyridine, pyrrole, pyrazine, pyrimidine, thiophene, thiazole, oxazole, isoxazole, furan, quinoline, pyrazole, and imidazole, optionally substituted with one or more substituents independently selected from C₁ to C₆ alkyl, halogen, haloalkyl, hydroxyl, C₁ to C₆ alkoxyl, amino, monoalkylamino, dialkylamino, thioalkyl, nitro, cyano, carboxyl, alkoxycarbonyl, aryl and heteroaryl.
14. The compound according to item 1, wherein the compound, or pharmaceutically acceptable salt thereof is of Formula 2, and selected from:

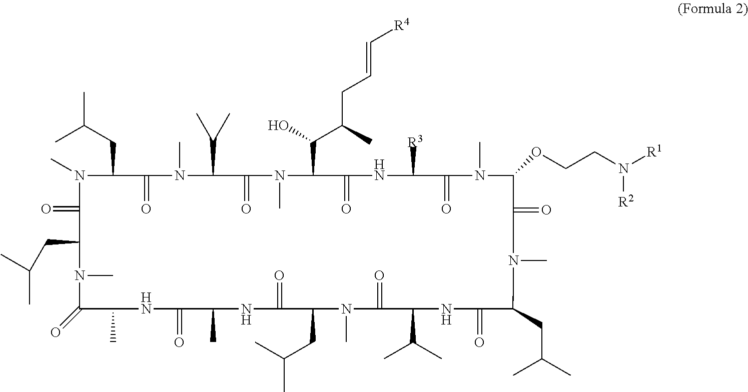

| Compound | R¹ | R² | R³ | R⁴ |
|---|---|---|---|---|
| 2 | —CH₃ | —CH₃ | —CH₂CH₃ | 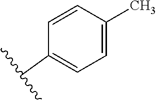 [SMILES: *c1ccc(C)cc1] |
| 3 | —CH₃ | —CH₃ | —CH₂CH₃ | 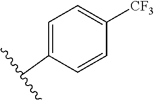 [SMILES: *c1ccc(C(F)(F)F)cc1] |
| 4 | —CH₃ | —CH₃ | —CH₂CH₃ | 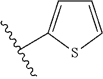 [SMILES: *c1cccs1] |
| 5 | —CH₃ | —CH₃ | —CH₂CH₃ | 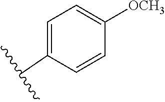 [SMILES: *c1ccc(OC)cc1] |
| 6 | —CH₃ | —CH₃ | —CH₂CH₃ | 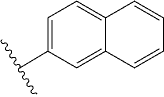 [SMILES: *c1ccc2ccccc2c1] |

15. Use of a compound or a pharmaceutical acceptable salt thereof as defined in any one of items 1-14 in the manufacture of a medicament, e.g. for the treatment of a disease or condition, e.g. a renal disease or condition.
16. Use of a compound or a pharmaceutical acceptable salt thereof as defined in any one items 1 to 14, in the manufacture of a medicament for the prevention or treatment of a cyclophilin-mediated disease or condition, e.g. a cyclophilin-mediated disease or condition of the kidney.
17. Use of a compound or a pharmaceutically acceptable salt thereof as defined in any one of items 1 to 14, in the manufacture of a medicament for the prevention or treatment of a disease or condition associated with cell injury or cell death.
18. The use according to items 15 to 17, wherein the disease or condition associated with cell injury or cell death is organ failure or organ injury.
19. The use according to item 18 wherein the organ is selected from the group consisting of kidney, liver, heart, lung, pancreas, intestine, cornea, skin, brain and nerve tissue.
20. The use according to any one of items 15 to 19, wherein the disease or condition is ischemia-reperfusion injury.
21. The use according to item 20, wherein the ischemia-reperfusion injury is renal ischemia-reperfusion injury.
22. The use according to any one of items 15 to 19, wherein the disease or condition is acute kidney injury.
23. The use according to item 22, wherein the acute kidney injury is associated with, or a consequence of kidney transplantation.
24. The use according to any one of items 15 to 23, wherein medicament is administered to an organ transplant recipient.
25. The use according to any one of items 15 to 24, wherein the medicament is adapted for oral administration, or is adapted for administration by intravenous injection or infusion.
26. The use according to any one of items 15-25 wherein the medicament is administered to an organ donor and/or to an organ recipient prior to, during, and/or after transplantation of an organ from said organ donor to said organ recipient.
27. Use of a compound or a pharmaceutically acceptable salt thereof as defined in any one of items 1-14 in the manufacture of a medicament for preserving an organ and/or protecting an organ from organ injury, the use comprising administering the compound to an organ donor prior to the removal of said organ from said donor and/or to an organ recipient prior to, during or after the transplantation of said organ.
28. Use of a compound or a pharmaceutically acceptable salt thereof as defined in any one of items 1-14 for preserving an organ and/or protecting an organ from organ injury, the use comprising administering the compound to an organ donor prior to removal of said organ from said donor, and/or to an organ.
29. A method of preserving or protecting an organ from organ injury, wherein the method comprises a step of administering a compound or a pharmaceutically acceptable salt thereof as defined in any one of items 1-14 to an organ donor prior to the removal of said organ from said donor, and/or to an organ.
30. The use or method according to any one of items 27-29, wherein the organ is selected from the group consisting of kidney, liver, heart, lung, pancreas, intestine, cornea, skin, brain and nerve tissue.
31. The use or method according to any one of items 27-30, wherein the donor is a live donor, or wherein the donor is a clinically dead donor.
32. The use or method according to any one of items 27-31, wherein the compound is administered to the donor and/or recipient by intravenous injection or infusion.
33. The use of a compound or a pharmaceutically acceptable salt thereof as defined according to any one of items 1-14 in the manufacture of a medicament for the prevention and/or treatment of a kidney condition or disease in a subject exposed to a nephrotoxin capable of inducing said kidney condition or disease, wherein the nephrotoxin is a nephrotoxic drug substance or an endogenous nephrotoxin.
34. The use according to item 33, wherein the nephrotoxic drug substance is selected from the group consisting of antimicrobial agents, cancer chemotherapeutic agents, blood pressure medicines including ACE inhibitors and angiotensin receptor blockers, macrolactone immunosuppressive agents, HIV protease inhibitors, peptic ulcer medicines, non-steroidal anti-inflammatory drugs, proton pump inhibitors, laxatives and contrast agents.
35. The use according to item 34, wherein the nephrotoxic drug substance is a chemotherapeutic agent is selected from the group consisting of platins (e.g. carboplatin, cisplatin, oxaliplatin or nedaplatin), anthracyclines (e.g. daunorubicin, doxorubicin, idarubicin, epirubicin), bleomycins, mitomycins, actinomycins, cyclophosphamides, cytarabine, capecitabine, gemcitabine, ifosfamide, interleukin-2, streptozocin, gemtuzumab ozogamicin, melphalan, methotrexate, pemetrexed, plicamycin, and trimetrexate.
36. The use according to item 35, wherein the subject is undergoing cancer treatment, wherein said cancer treatment comprises the administration of the chemotherapeutic agent to the subject.
37. The use according to item 34, wherein the nephrotoxic drug substance is an antimicrobial agent is selected from the group consisting of, aminoglycosides (e.g. gentamicin, tobramycin, amikacin, netilmicin, apramicin, streptomycin, kanamycin, neomycin, sisomycin), beta-lactams (e.g tazobactam, or piperacillin/tazobactam), polypeptide antibiotics (e.g. polymyxins such as polymyxin A, B, C, D, E (colistin), glycopeptide antibiotics (e.g. vancomycin), outer membrane protein targeting antibiotics, (e.g. murepavadin), antifungal agent (e.g. amphotericin B) and combinations thereof.
38. The use according to item 37, wherein the antimicrobial agent is gentamicin.
39. The use according to any one of items 33 to 38, wherein the subject is suffering from an infection, and wherein said infection is treated by administering the antimicrobial agent to the subject.
40. The use according to item 34, wherein the blood pressure medicine is an ACE inhibitor, optionally selected from the group consisting of captopril, benazepril, enalapril, fosinopril, and ramipril; or an angiotensin receptor blocker, optionally selected from the group consisting of candesartan, valsartan, irbesartan, olmesartan, telmisartan, eprosartan, and losartan.
41. The use according to item 34, wherein the HIV protease inhibitor is selected from the group consisting of indinavir and ritonavir.
42. The use according to item 34, wherein the peptic ulcer medicine is selected from the group consisting of cimetidine, esomeprazole, lansoprazole, omeprazole, pantoprazole, and rabeprazole.
43. The use according to item 34, wherein the non-steroidal anti-inflammatory drug is selected from the group consisting of ibuprofen, ketoprofen, diclofenac, and aspirin.
44. The use according to item 34, wherein the laxative is selected from sodium phosphate.
45. The use according to item 34, wherein the nephrotoxic drug substance is a contrast agent, optionally an iodinated contrast agent (e.g. iothalamate, or iodixanol, or iohexol).
46. The use according to item 33, wherein the endogenous nephrotoxin is myoglobin; and optionally wherein the subject has a creatine phosphokinase serum level of at least 5 times greater than baseline.
47. The use according to any one of items 46, wherein the subject has experienced or is suffering from physical trauma or crush injury, exposure to electrical current, extreme physical exertion or activity, and temperature extremes.
48. The use according to any one of items 46 or 47, wherein the medicament is administered to the subject prior to exposure to, or engagement with activities (e.g. extreme physical activity) associated with or at risk for onset of rhabdomyolysis.
49. The use according to any one of items 33 to 45, wherein the nephrotoxic drug substance is administered to the subject repeatedly; optionally wherein the nephrotoxic drug substance is administered at least twice, optionally at least once daily over a period of at least 3 days, or 7 days.
50. The use according to any one of items 33 to 49, wherein the kidney condition or disease is nephrotoxin-induced acute kidney injury or kidney failure.
51. The use according to any one of items 33 to 50, wherein the kidney condition or disease is selected from rhabdomyolysis, hemolysis, myoglobinuria, or optionally tumour lysis or myeloma-induced acute kidney injury.
52. The use according to any one of items 33 to 51, wherein the subject has a pre-existing condition or disease that increases the subject's risk of developing a kidney condition or disease when exposed to the nephrotoxin, optionally wherein said pre-existing kidney condition is chronic kidney disease; further optionally wherein the subject has a history of renal impairment or requires dialysis.
53. The use according to item 52, wherein the subject has reduced renal function, optionally wherein the subject has blood urea nitrogen levels of at least 1.5 to 3 times higher than baseline, and/or serum creatinine levels at least 1.5 to 3 times higher than baseline, and/or oliguria.
54. The use according to any one of items 33 to 53, wherein the medicament is administered to the subject prior to the subject's exposure to the nephrotoxic drug substance; optionally wherein a dose of the medicament is administered to the subject within 24 hours or less before a dose of the nephrotoxic drug substance is administered to the subject, further optionally wherein a dose of the medicament is administered to the subject within about 6 hours or less, and optionally within about 2 hours or less, before the nephrotoxic drug substance is administered to the subject.
55. The use according to any one of items 33 to 54, wherein the medicament is administered to the subject after the onset of reduced renal functions as characterized by any one or combination of: blood urea nitrogen levels of at least 1.5 to 3 times higher than baseline, serum creatinine levels of at least 1.5 to 3 times higher than baseline, and oliguria.
56. The use according to item 55, wherein a dose of the medicament is administered to the subject 1 to 24 hours after the onset of reduced renal function.
57. The use according to any one of items 33 to 56, wherein the medicament is administered repeatedly to a subject during a first period of time which commences before and overlaps with a second period of time wherein the subject is exposed to repeatedly to the nephrotoxic drug substance.
58. Use of Compound I or a pharmaceutically acceptable salt thereof as defined in item 1 in the manufacture of a medicament for the prevention and/or reduction of blood urea nitrogen levels of at least 1.5 to 3 times higher than baseline and/or serum creatinine levels of at least 1.5 to 3 times higher than baseline in a subject exposed to a nephrotoxin.
59. The use according to item 58, wherein the nephrotoxin is as defined in any one of items 33 to 35, 37 to 38, or items 40 to 46.
60. The use according to item 58 or 59, wherein the subject is defined as in any one of items 33, 36, 39, 46 to 47, 52 or 53.
61. The use according to items 58 to 60, wherein the medicament is administered as defined in any one of items 48 to 51, or items 54 to 57.
62. The use according to any one of the preceding items, wherein the recipient, donor, and/or subject is a human.
63. The use according to any one of the preceding items, wherein the medicament is adapted or formulated for administration by infusion or by injection, preferably subcutaneous, intramuscular or intravenous injection or intravenous or subcutaneous infusion; or is adapted or formulated for oral administration.
64. A compound or a pharmaceutically acceptable salt thereof as defined in any one of items 1 to 14, for use as a medicine, optionally for use as a medicine for the treatment of a disease or condition e.g. a renal disease or condition.
65. A compound for use according to item 64 for use as a cyclophilin inhibitor (e.g. cyclophilin A and/or cyclophilin D, or preferably cyclophilin D); preferably for use in the prevention and/or treatment of a cyclophilin-mediated disease or condition, e.g. a cyclophilin-mediated disease or condition of the kidney.
66. A compound for use according to item 64 or 65, wherein the use comprises the prevention and/or treatment of a disease or condition associated with cell injury or cell death.
67. The compound for use according to item 66, further comprising any one or combination of the features of items 18 to 26.
68. A compound or a pharmaceutically acceptable salt thereof as defined in any one of items 1-14 for use in the preservation of an organ and/or protecting an organ from organ injury, the use comprising administering the compound to an organ donor prior to the removal of said organ from said donor and/or to an organ recipient prior to, during or after the transplantation of said organ.
69. The compound for use according to item 76, further comprising any one or any combination of the features as defined for items 30 to 32.
70. A compound or a pharmaceutically acceptable salt thereof as defined according to any one of items 1-14 for use in the prevention and/or treatment of a kidney condition or disease in a subject exposed to a nephrotoxin capable of inducing said kidney condition or disease, wherein the nephrotoxin is a nephrotoxic drug substance or an endogenous nephrotoxin.
71. The compound for use according to claim 80, further comprising any one or any combination of the features as defined in items 33 to 63.
72. A method for treating a disease or condition comprising administering a compound or a pharmaceutically acceptable salt thereof as defined in any one of items 1 to 14, to a subject in need thereof; for example, wherein the disease or a condition of the kidney.
73. A method for inhibiting cyclophilin or for treating or preventing a cyclophilin-mediated disease or condition by inhibition of cyclophilin, comprising administering a compound or a pharmaceutically acceptable salt thereof as defined in any one of items 1 to 14 to a subject in need thereof, optionally wherein the cyclophilin is cyclophilin A and/or wherein the cyclophilin is cyclophilin D.
74. The method according to item 72 or 73, wherein the disease or condition is associated with cell injury or cell death.
75. The method according to item 74, further comprising any one or combination of the features as defined in items 18 to 26.
76. A method for preserving an organ and/or protecting an organ from organ injury, comprising administering a compound or a pharmaceutically acceptable salt thereof as defined in any one of items 1-14 to an organ donor prior to the removal of said organ from said donor and/or to an organ recipient prior to, during or after the transplantation of said organ.
77. The method according to item 76, further comprising any one or any combination of the features as defined for items 30 to 32
78. A method for preventing and/or treating a kidney condition or disease in a subject exposed to a nephrotoxin capable of inducing said kidney condition or disease, wherein the nephrotoxin is a nephrotoxic drug substance or an endogenous nephrotoxin and wherein the method comprises administering to said subject a compound according any one of items 1-14, or a pharmaceutically acceptable salt thereof.
79. The method according to item 98, wherein the method comprises any one or combination of the features as defined in items 33 to 63.
80. A pharmaceutical composition comprising a compound or a pharmaceutically acceptable salt thereof as defined in any one of items 1 to 14, and one or more pharmaceutically acceptable excipients.
81. The use of a pharmaceutical composition according to item 80 in the prevention and/or treatment of a disease or condition as described in any one or combination of the preceding items, or in the manufacture of a medicament for use in the prevention and/or treatment of a disease or condition as described in any one or combination of the above items.
82. A pharmaceutical composition according to item 80 for use in accordance with any one or combination of the uses described in any one of the preceding items.
83. A method, e.g. a method of prevention and/or treatment, or a method of preservation and/or protection as described in any one of the preceding items, wherein the method comprises administering a pharmaceutical composition according to item 80 to a subject in need thereof.
84. A compound of Formula 1, or a pharmaceutically acceptable salt thereof, wherein R¹, R², R³ and R⁴ are as defined in any one of the features or combinations described in items 1 to 14, and wherein one or more hydrogen atoms of the —(CH₂)ₙ— moiety of the amino alkoxy sarcosine substituent is independently replaced with a substituent, e.g. an alkyl substituent (e.g. methyl) or another substituent as described herein.
85. Use of a compound according to item 84 for the manufacture of a medicament according to any one or combination of features as defined in items 15 to 63.
86. A process for the preparation of any one of the compounds of Formula 1 or 2 as defined in items 1 to 14 or a compound as defined in item 84, the process comprising a step a) of reacting a cyclosporin A intermediate e.g. a thiopyridyl intermediate, with an amino alcohol compound, and optionally, copper triflate.
87. The process of item 86, wherein the amino alcohol is a compound of Formula

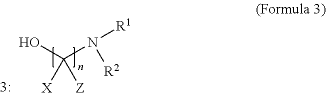

wherein the substituents, X and Z may be independently selected from H, alkyl (e.g. C₁-C₆ alkyl, for example methyl), substituted alkyl, e.g. substituted C₁ to C₆ alkyl or wherein X and Z may be joined together to form a C₃ to C₆ cycloalkyl or heterocycloalkyl ring; and wherein the integer n, R¹, R² are as defined in any one or combination of features defined in items 1-14.
88. The process of item 86-87, comprising the reaction of a compound obtained from step a), the reaction comprising an alkenyl or vinyl compound and a catalyst e.g. Grubbs catalyst.

The following examples serve to illustrate the invention, however should not to be understood as restricting the scope of the invention.

### EXAMPLES

### Example 1—Preparation of Compounds

Compounds 2 to 6, such as described herein above may be prepared from Compound 1 described above. The preparation of Compound 1 is, for example, described in WO2019/016572 A1.

Preparation of Compound 2

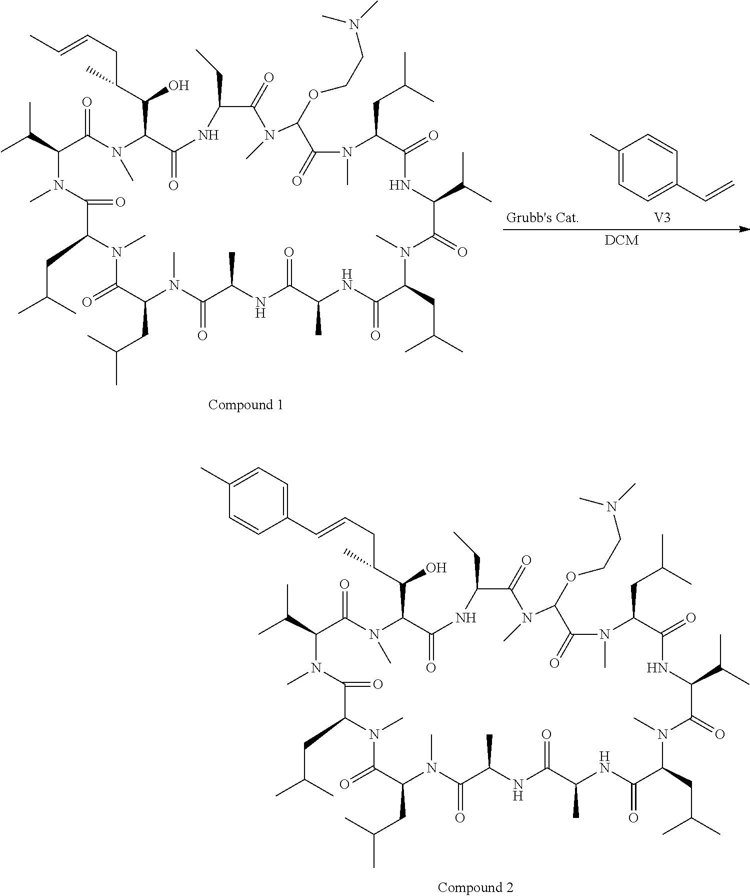

Grubbs catalyst (20 mg, 0.0236 mmol) was added to a solution of Compound 1 (50 mg, 0.0388 mmol) in 1.5 mL DCM. The olefin V3 (92 mg, 0.7760 mmol) was added dropwise to the mixture at room temperature. The resultant mixture was stirred at 40° C. for 16 hours. After cooled to room temperature, the mixture was filtered. The filtrate was concentrated and purified by chromatography to give the desired product Compound 2 (¹H-NMR (400 MHz CDCl₃, δ (ppm)): 5.99, sarcosine residue); HRMS Electrospray (M+1) 1366.6; 1367.6); mass according to isotope distribution (1364.94 (100%), 1365.94 (77.9%)).

Compound 3

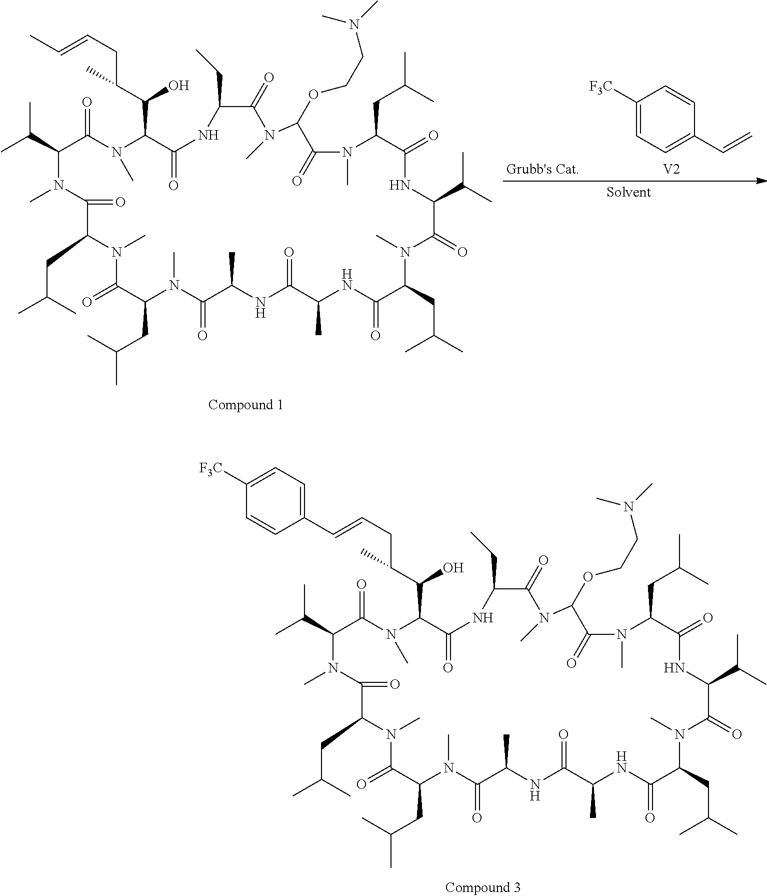

Grubbs catalyst (100 mg, 0.118 mmol) was added to a solution of Compound 1 (200 mg, 0.155 mmol) in 1.5 mL CCl₄. The olefin V2 (534 mg, 3.101 mmol) was added dropwise to the mixture at 70° C. The resultant mixture was stirred at 70° C. for 48 hours. After cooling to room temperature, the mixture was filtered. The filtrate was concentrated and purified by column chromatography to give the desired product Compound 3 (¹H-NMR (400 MHz CDCl₃, δ (ppm)): 6.17, sarcosine residue); HRMS Electrospray (M+1) 1420.70; 1422.25; mass according to isotope distribution 1418.91 (100%), 1419.92 (77.9%)).

Compound 4

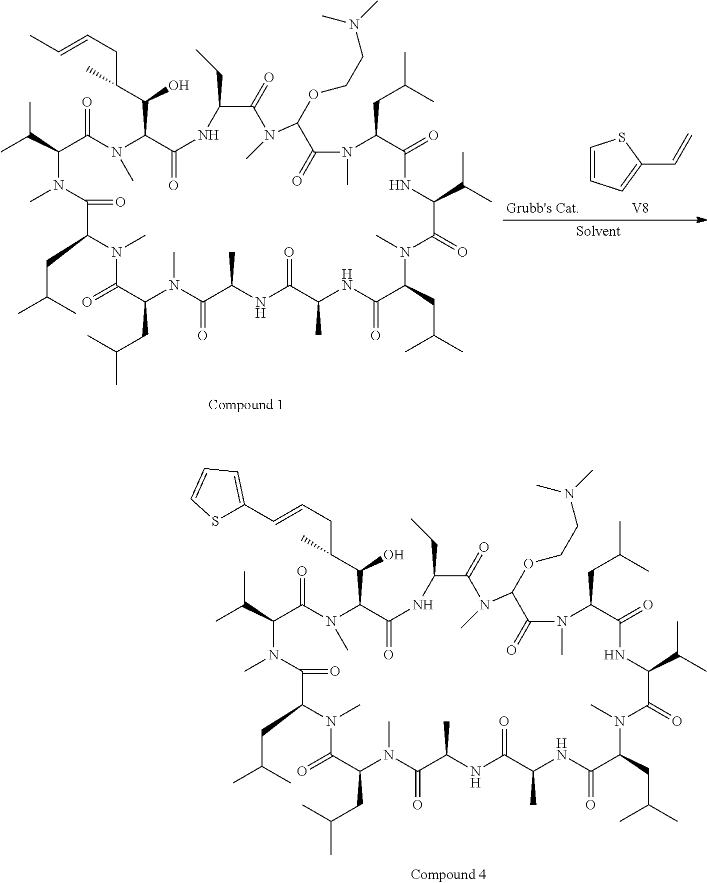

Grubbs catalyst (20 mg, 0.0236 mmol) was added to a solution of Compound 1 (50 mg, 0.0388 mmol) in 1.5 mL DCM. The olefin V8 (85 mg, 0.7753 mmol) was added dropwise to the mixture at room temperature. The mixture was stirred at 40° C. for 16 hours. After cooling to room temperature, the mixture was filtered. The filtrate was concentrated and purified by chromatography to give the desired product Compound 4 ((¹H-NMR (400 MHz CDCl₃, δ (ppm)): 5.99, sarcosine residue); HRMS Electrospray (M+1) 1358.5; 1359.4; mass according to isotope distribution, 1356.88 (100%), 1357.89 (74.6%)).

Compound 5

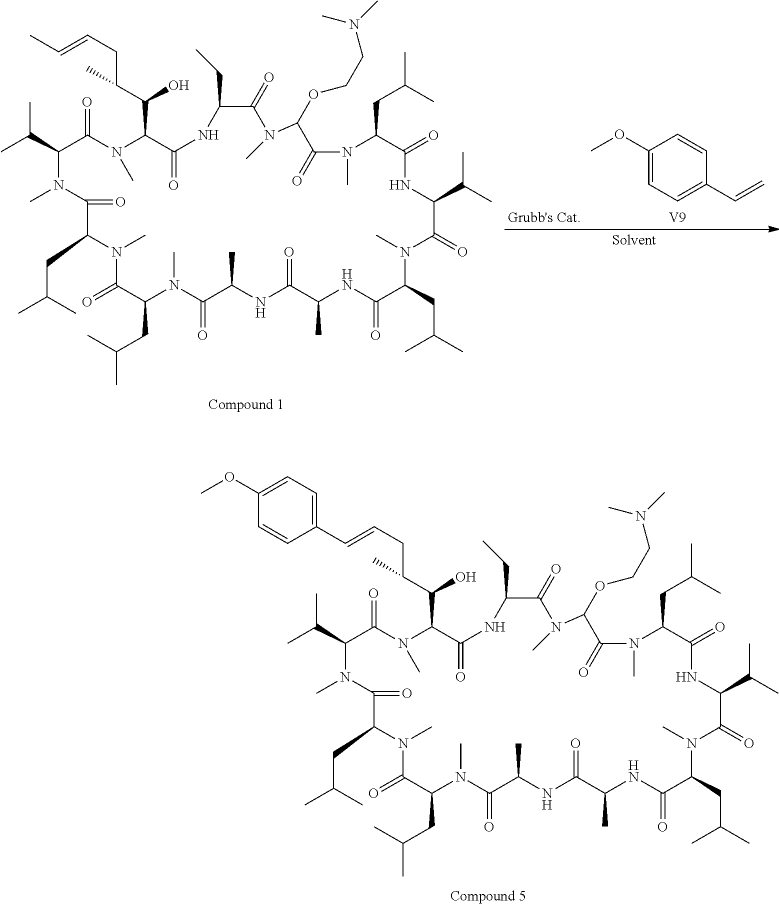

The Grubbs catalyst (20 mg, 0.0236 mmol) was added to a solution of Compound 1 (50 mg, 0.0388 mmol) in 1.5 mL DCM at room temperature. The olefin V9 (104 mg, 0.7753 mmol, 20.0 eq) was added dropwise to the mixture. The mixture was stirred at 40° C. for 16 hours. After cooled to room temperature, the mixture was filtered. The filtrate was concentrated and purified by chromatography to give the desired product Compound 5 ((¹H-NMR (400 MHz CDCl₃, δ (ppm)): 5.99, sarcosine residue); HRMS Electrospray (M+1) 1382.6; 1383.6; mass according to isotope distribution 1380.94 (100%), 1381.94 (77.9%)).

Compound 6

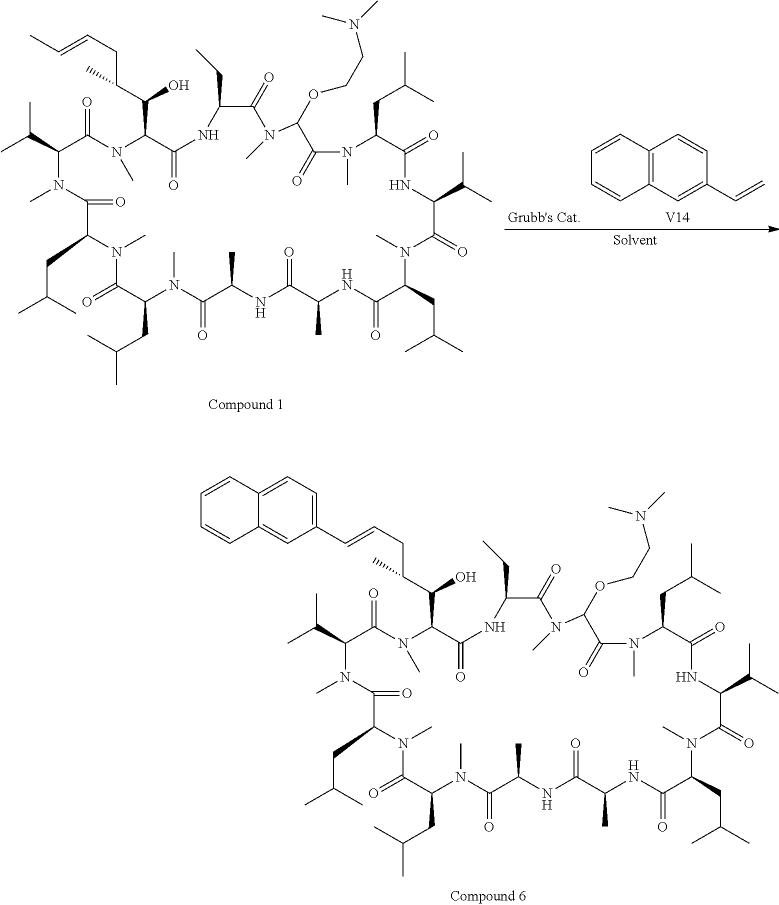

The Grubbs catalyst (400 mg, 0.466 mmol) was added to a solution of Compound 1 (500 mg, 0.388 mmol) in 10 mL CCl₄. A solution of the olefin V14 (1.2 g, 7.753 mmol) in 5 mL CCl₄ was added dropwise to the mixture. The mixture was stirred at 40° C. for 16 hours. After cooled to room temperature, the mixture was filtered. The filtrate was concentrated and purified by chromatography to give the desired product Compound 6 (¹H-NMR (CDCl₃): δ (ppm) 5.95, sarcosine residue); HRMS Electrospray (M+1) 1402.10; 1403.15; calculated mass according to isotope distribution 1400.94 (100%) 1401.94 (81.1%)).

### Example 2—Functional and Inhibitional Assays

Cyclophilin A and D peptidyl-prolyl isomerase functional assays using human recombinant enzymes (PPlase assay), as well as in a calcineurin inhibition assay with and without cyclophilin A are conducted. The compounds are also tested in a calcium retention capacity (CRC) assay in permeabilized HepG2. Cyclosporin A is used as a control in all assays.

The compounds are supplied as a dry powder or oils and made up as a 10 mM stock solution in 100% DMSO. Subsequent dilutions were made in 100% DMSO for use in all assays.

Cyclophilin Peptidyl-Prolyl Isomerase Functional Assay

Measurements are performed using an Agilent 8453 spectrophotometer. Assay buffer was cooled to 10° C. (with stirring) in a precision glass cuvette and inhibitor is added from a DMSO stock solution to afford a final concentration of <1% DMSO. A blank spectrum is obtained and then enzyme and substrate are added and the change in absorbance measured over 5 min. A first order rate is fitted to the absorbance data to obtain a rate constant (first 10 to 15 s were eliminated due to mixing). The catalytic rate is calculated from the enzymatic rate minus the background rate. The enzymatic rate constant, determined in duplicate at each inhibitor concentration, is plotted against inhibitor concentration and a non-linear fit by SigmaPlot generated a Kᵢ.

Calcineurin Phosphatase Inhibition Assay With and Without Cyclophilin A

This colorimetric 96 well assay is designed for inhibitor screening of recombinant Calcineurin (CaN). Activity is determined using the RII phosphopeptide substrate, the most efficient and selective peptide known for calcineurin, and detection of free phosphate released is based on the classic malachite green assay. CypA and CsA form a complex which binds CaN/calmodulin, which will inhibit dephosphorylation of the RII peptide. In the presence of recombinant CypA, cyclosporine-like cyclophilin inhibitors were screened in the assay to determine inhibition of calcineurin phosphatase activity. In a 96-well plate, two dilution series are prepared, one with the cyclophilin A enzyme (7-point) and another without (4-point). An assay buffer/calcineurin/calmodulin master mix is added followed by the phosphopeptide substrate (RII). After incubation at 30° C. the reaction is stopped by the addition of malachite green/molybdate reagent. The coloured complex formed with liberated phosphate is quantified by reading the absorbance at 620 nm. The blank corrected data was plotted against inhibitor concentration to determine an IC₅₀ value.

Calcium Retention Capacity (CRC) Assay in Permeabilized HepG2

HepG2 cells are permeabilised with 100 μM digitonin for 10 min in ice cold buffer containing 1 mM EGTA. Following two wash steps to remove the digitonin, the cells are plated into 96 well black and clear plates at 1e⁶ cell per well in 180 μL assay buffer containing 0.5 μM Calcium Green 5N. Compounds dilutions are made in DMSO to 1000-fold the final concentration, diluted 1:100 in assay buffer and added to the assay as 20 μL per well. The assay buffer contained 5 mM glutamate and 2.5 mM malate. The cell plate is immediately run on the FLIPR Tetra™ which added 5 μL of 200 μM (5 μM) calcium chloride every 5 minutes whilst reading the plate every 3 seconds. The area under the curve at each concentration of compound is calculated. EC₅₀ values are calculated.

These assays were conducted on Compound 2, as well as Reference Compound 1 and Cyclosporin A as a control. Results are summarized in the Tables 1-3 below:

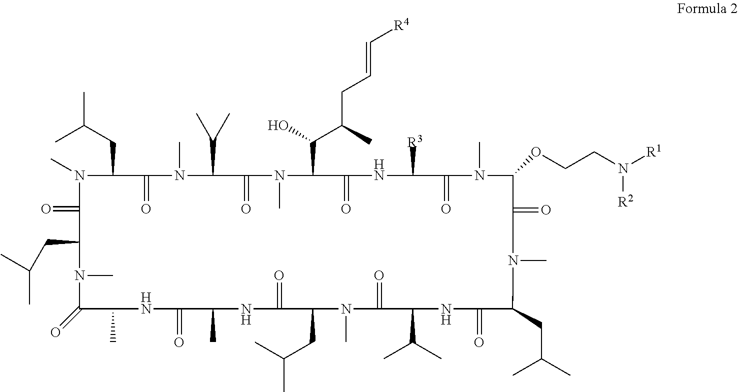

TABLE 1
Cyclophilin Peptidyl-Prolyl Isomerase Functional Assay Results

| | | | | | Inhibition | Inhibition |
|---|---|---|---|---|---|---|
| | | | | | (Kᵢ, nM) | (Kᵢ, nM) |
| | | | | | Human | Human |
| Compound | R¹ | R² | R³ | R⁴ | CypA | CypD |
| Cyclosporin A | — | — | — | — | 7.8 | 9.1 |
| Reference | —CH₃ | —CH₃ | —CH₂CH₃ | —CH₃ | 4.1 | 12 |
| Compound 1 | | | | | | |
| 2 | —CH₃ | —CH₃ | —CH₂CH₃ | 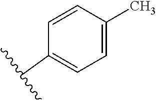 [SMILES: *c1ccc(C)cc1] | 6 | 11 |
| 3 | —CH₃ | —CH₃ | —CH₂CH₃ | 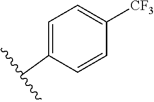 [SMILES: *c1ccc(C(F)(F)F)cc1] | 25 | 9.4 |
| 4 | —CH₃ | —CH₃ | —CH₂CH₃ | 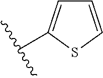 [SMILES: *c1cccs1] | 13 | 11 |
| 5 | —CH₃ | —CH₃ | —CH₂CH₃ | 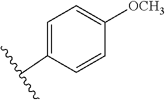 [SMILES: *c1ccc(OC)cc1] | 5.5 | 8.4 |
| 6 | —CH₃ | —CH₃ | —CH₂CH₃ | 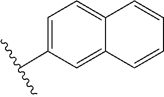 [SMILES: *c1ccc2ccccc2c1] | 13 | 6.5 |

TABLE 2
Calcineurin Phosphatase Inhibition Assay With
and Without Cyclophilin A-Results

| | | | | | Calcineurin | Calcineurin |
|---|---|---|---|---|---|---|
| | | | | | Inhibition | Inhibition |
| | | | | | (IC₅₀, nM) | (IC₅₀, nM) |
| Compound | R¹ | R² | R³ | R⁴ | +CypA | −CypA |
| Cyclosporin A | — | — | — | — | 107 | >10000 |
| Reference | —CH₃ | —CH₃ | —CH₂CH₃ | —CH₃ | 1551 | 2276 |
| Compound 1 | | | | | | |
| 2 | —CH₃ | —CH₃ | —CH₂CH₃ | 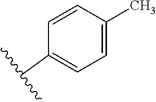 [SMILES: *c1ccc(C)cc1] | 1852 | 3952 |
| 3 | —CH₃ | —CH₃ | —CH₂CH₃ | 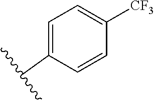 [SMILES: *c1ccc(C(F)(F)F)cc1] | 6367 | >10000 |
| 4 | —CH₃ | —CH₃ | —CH₂CH₃ | 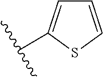 [SMILES: *c1cccs1] | 817 | 1158 |
| 5 | —CH₃ | —CH₃ | —CH₂CH₃ | 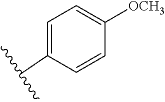 [SMILES: *c1ccc(OC)cc1] | 2426 | 2658 |
| 6 | —CH₃ | —CH₃ | —CH₂CH₃ | 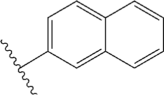 [SMILES: *c1ccc2ccccc2c1] | 6429 | >10000 |

TABLE 3
Calcium Retention Capacity (CRC) Assay in permeabilized HepG2-Results

| | | | | | Calcium |
|---|---|---|---|---|---|
| | | | | | Retention |
| | | | | | Capacity |
| Compound | R¹ | R² | R³ | R⁴ | EC₅₀ (nM) |
| Cyclosporin A | — | — | — | — | 369 |
| Reference | —CH₃ | —CH₃ | —CH₂CH₃ | —CH₃ | 916 |
| Compound 1 | | | | | |
| 2 | —CH₃ | —CH₃ | —CH₂CH₃ | 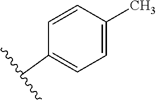 [SMILES: *c1ccc(C)cc1] | 512 |
| 3 | —CH₃ | —CH₃ | —CH₂CH₃ | 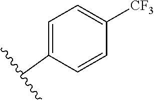 [SMILES: *c1ccc(C(F)(F)F)cc1] | 1371 |
| 4 | —CH₃ | —CH₃ | —CH₂CH₃ | 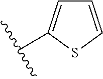 [SMILES: *c1cccs1] | 867 |
| 5 | —CH₃ | —CH₃ | —CH₂CH₃ | 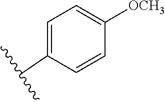 [SMILES: *c1ccc(OC)cc1] | 623 |
| 6 | —CH₃ | —CH₃ | —CH₂CH₃ | 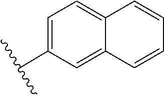 [SMILES: *c1ccc2ccccc2c1] | 964 |

It was observed (ref. Table 1) that the tested compounds are generally effective as inhibitor of human cyclophilin, in particular the inhibition of human cyclophilin D. Inhibition of cyclophilin D is generally observed to be improved compared to the cyclosporin A control, and also at levels comparable to reference Compound 1.

As shown in Table 2, calcineurin inhibition activity of the tested compounds in the presence or absence of cyclophilin A were generally lower compared to the control cyclosporin A and reference Compound 1.

The binding to cyclophilin A and inhibition of calcineurin is closely linked to immunosuppression. Without wishing to be bound by theory, it is believed that cell injury or death, e.g. cell necrosis and associated diseases or conditions are driven by inflammation processes, the prevention or treatment of which may be better achieved by compounds having potent anti-inflammatory activity but lower or decreased level of immunosuppressive activity, such as characterized by decreased binding to cyclophilin A/calcineurin.

In particular, it is believed that increased inhibition of especially of cyclophilin D, which regulates the opening of the mitochondrial permeability transition pore (PTP) may lead to improved treatment and/or prevention of cell injury or cell death and accordingly, in the prevention or treatment of associated conditions or diseases.

The calcium retention capacity (CRC) assay is a model for mitochondrial function, based on the inhibition of opening of the mitochondrial permeability transition pore. Ca++ overload over a sustained period of time is understood to trigger prolonged PTP opening and mitochondrial dysfunction, leading to cell death. The assay as described above measures the loss of inhibition based on the release of calcium, which is determined by increased intensity of fluorescence reporting of the calcium-binding dye used in the assay. It was observed that the compounds tested have increased calcium retention capacity compared to the cyclosporin A control (Table 3).

1. A compound of Formula 1, or a pharmaceutically acceptable salt thereof, 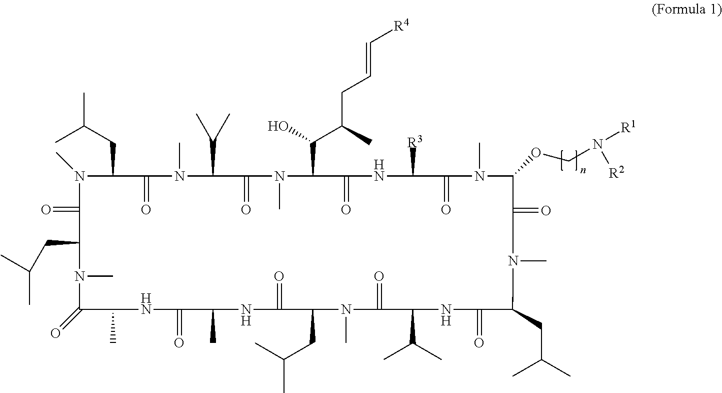
wherein:
n is selected from an integer between 2 and 5;
R¹ and R² are independently selected from H, C₁ to C₆ alkyl or wherein R¹ and
R² may be joined together to form a C₃ to C₆ cycloalkyl or heterocycloalkyl ring;
R³ is ethyl, 1-hydroxyethyl, isopropyl, or n-propyl; and
wherein R⁴ is aryl, substituted aryl, heteroaryl and substituted heteroaryl, and
wherein the substitution is optionally one or more substituents independently selected from C₁ to C₆ alkyl, halogen, haloalkyl, hydroxyl, C₁ to C₆ alkoxyl, amino, monoalkylamino, dialkylamino, thioalkyl, nitro, cyano, carboxyl, alkoxycarbonyl, aryl and heteroaryl.

2. The compound according to claim 1, wherein at least one of R¹ or R² is C₁ to C₆ alkyl.

3. The compound according to claim 2 wherein R¹ and R² are both —CH₃.

4. The compound according to claim 1, wherein R¹ and R² are joined together to form a C₃ to C₆ cycloalkyl or heterocycloalkyl ring.

5. The compound according to claim 4 wherein R¹ and R² are joined together to form a morpholinyl residue.

6. The compound according to any one of the preceding claims, wherein n is 2.

7. The compound according to any one of the preceding claims, wherein R³ is ethyl.

8. The compound according to any one of the preceding claims, wherein R³ is isopropyl, n-propyl, or 1-hydroxyethyl.

9. The compound according to any one of the preceding claims, wherein R⁴ is aryl or substituted aryl.

10. The compound according to any one of the preceding claims, wherein R⁴ is phenyl, or substituted phenyl, wherein the substitution is optionally one or more substituents independently selected from C₁ to C₆ alkyl (e.g. methyl, or t-butyl), halogen (e.g. chloro, fluoro, bromo, or iodo), haloalkyl (e.g. trifluoromethyl), hydroxyl, C₁ to C₆ alkoxyl (e.g. methoxy, phenoxy, or t-butoxy), amino, monoalkylamino, dialkylamino (e.g. dimethylamino), thioalkyl, nitro, cyano, carboxyl ((e.g. —COOCH₃), alkoxycarbonyl (e.g. acetoxy), aryl (e.g. phenyl) and heteroaryl.

11. The compound according to any one of claims 1 to 9, wherein R⁴ is naphthalene, or substituted naphthalene.

12. The compound according to any one of claims 1 to 8, wherein R⁴ is heteroaryl or substituted heteroaryl.

13. The compound according to claim 12, wherein the R⁴ is selected from the group consisting of pyridine, pyrrole, pyrazine, pyrimidine, thiophene, thiazole, oxazole, isoxazole, furan, quinoline, pyrazole, and imidazole, optionally substituted with one or more substituents independently selected from C₁ to C₆ alkyl, halogen, haloalkyl, hydroxyl, C₁ to C₆ alkoxyl, amino, monoalkylamino, dialkylamino, thioalkyl, nitro, cyano, carboxyl, alkoxycarbonyl, aryl and heteroaryl.

14. The compound according to claim 1, wherein the compound, or pharmaceutically acceptable salt thereof is of Formula 2, and selected from:

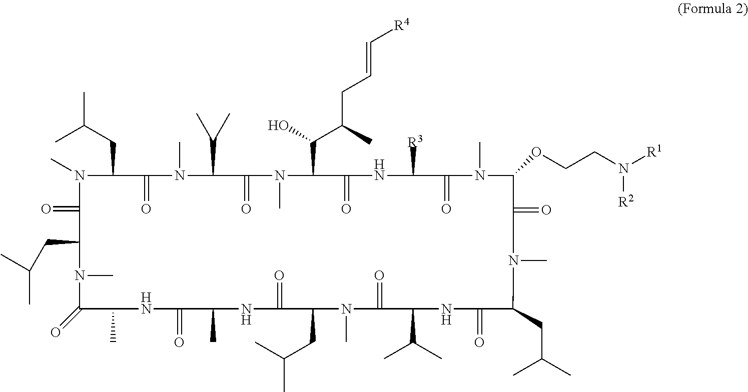

| Compound | R¹ | R² | R³ | R⁴ |
|---|---|---|---|---|
| 2 | —CH₃ | —CH₃ | —CH₂CH₃ | 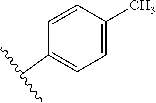 [SMILES: *c1ccc(C)cc1] |
| 3 | —CH₃ | —CH₃ | —CH₂CH₃ | 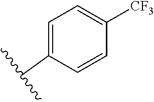 [SMILES: *c1ccc(C(F)(F)F)cc1] |
| 4 | —CH₃ | —CH₃ | —CH₂CH₃ | 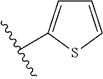 [SMILES: *c1cccs1] |
| 5 | —CH₃ | —CH₃ | —CH₂CH₃ | 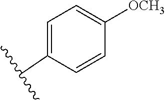 [SMILES: *c1ccc(OC)cc1] |
| 6 | —CH₃ | —CH₃ | —CH₂CH₃ | 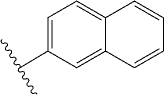 [SMILES: *c1ccc2ccccc2c1] |

15. Use of a compound or a pharmaceutical acceptable salt thereof as defined in any one of claims 1-14 in the manufacture of a medicament.

16. Use of a compound or a pharmaceutical acceptable salt thereof as defined in any one of claims 1 to 14, in the manufacture of a medicament for: a) the prevention and/or treatment of a cyclophilin-mediated disease or condition and/or b) the prevention and/or treatment of a renal disease or condition.

17. Use of a compound or a pharmaceutically acceptable salt thereof as defined in any one of claims 1 to 14, in the manufacture of a medicament for the prevention and/or treatment of a disease or condition associated with cell injury or cell death.

18. The use according to claim 17, wherein the disease or condition associated with cell injury or cell death is organ failure or organ injury.

19. The use according to claim 18 wherein the organ is selected from the group consisting of kidney, liver, heart, lung, pancreas, intestine, cornea, skin, brain and nerve tissue; preferably wherein the organ is selected from the kidney.

20. The use according to any one of claims 15 to 19, wherein the disease or condition is ischemia-reperfusion injury.

21. The use according to claim 20, wherein the ischemia-reperfusion injury is renal ischemia-reperfusion injury.

22. The use according to any one of claims 15 to 19, wherein the disease or condition is acute kidney injury.

23. The use according to claim 22, wherein the acute kidney injury is associated with, or a consequence of kidney transplantation.

24. The use according to any one of claims 15 to 23, wherein medicament is administered to an organ transplant recipient.

25. The use according to any one of claims 15 to 24, wherein the medicament is adapted for oral administration, or is adapted for administration by intravenous injection or infusion.

26. The use according to any one of claims 15-25 wherein the medicament is administered to an organ donor and/or to an organ recipient prior to, during, and/or after transplantation of an organ from said organ donor to said organ recipient.

27. Use of a compound or a pharmaceutically acceptable salt thereof as defined in any one of claims 1-14 in the manufacture of a medicament for preserving an organ and/or protecting an organ from organ injury, the use comprising administering the compound to an organ donor prior to the removal of said organ from said donor and/or to an organ recipient prior to, during or after the transplantation of said organ.

28. Use of a compound or a pharmaceutically acceptable salt thereof as defined in any one of claims 1-14 for preserving an organ and/or protecting an organ from organ injury, the use comprising administering the compound to an organ donor prior to removal of said organ from said donor, and/or to an organ.

29. A method of preserving or protecting an organ from organ injury, wherein the method comprises a step of administering a compound or a pharmaceutically acceptable salt thereof as defined in any one of claims 1-14 to an organ donor prior to the removal of said organ from said donor, and/or to an organ.

30. The use or method according to any one of claims 27-29, wherein the organ is selected from the group consisting of kidney, liver, heart, lung, pancreas, intestine, cornea, skin, brain and nerve tissue.

31. The use or method according to any one of claims 27-30, wherein the donor is a live donor, or wherein the donor is a clinically dead donor.

32. The use or method according to any one of claims 27-31, wherein the compound is administered to the donor and/or recipient by intravenous injection or infusion.
